(19) United States
(12) Patent Application Publication
(10) Pub. No.: US 2024/0424083 A1
(43) Pub. Date: Dec. 26, 2024

(54) CORONAVIRUSES, VACCINES COMPRISING THE SAME, AND METHODS FOR PREVENTING DISEASE

(71) Applicant: LOYOLA UNIVERSITY OF CHICAGO, Chicago, IL (US)

(72) Inventors: SUSAN BAKER, Elmhurst, IL (US); Xufang Deng, Lyons, IL (US)

(21) Appl. No.: 18/341,330

(22) Filed: Jun. 26, 2023

(51) Int. Cl. A61K 39/215; A61P 31/14; C12N 7/00

(52) U.S. Cl. CPC A61K 39/215; A61P 31/14; C12N 7/00; A61K 2039/5254

(57) ABSTRACT

Coronaviruses, vaccines comprising the same, and methods for preventing disease. One embodiment of such includes a live, attenuated coronavirus comprising a variant replicase gene encoding polyproteins comprising a non-structural protein (nsp)-15, the replicase gene encoding the nsp15 and causes any change, including mutations and/or deletions, that affects the stability or activity of the nsp15.

### CROSS REFERENCE TO RELATED APPLICATIONS

This application claims the benefit of U.S. Provisional Application No. 62/466,779, filed Mar. 3, 2017 the contents of which are incorporated herein by reference.

### STATEMENT REGARDING FEDERALLY SPONSORED RESEARCH

This invention was made with government support under Grant or Contract No. R01 A1085089 awarded by the National Institutes of Health, and Agricultural Research Service Project 5030-32000-118-11S awarded by the United States Department of Agriculture. The government has certain rights in the invention.

### BACKGROUND OF THE INVENTION

The present invention generally relates to coronaviruses. The invention particularly relates to vaccines and methods of producing the same for existing and emerging coronaviruses.

Coronaviruses are species of viruses belonging to the subfamily Coronavirinae in the family Coronaviridae, and are positive-sense RNA viruses that infect humans and animals and cause respiratory, gastrointestinal or neurologic disease. Coronaviruses can emerge from animal reservoirs to cause significant epidemics in humans, exemplified by Severe Acute Respiratory Syndrome coronavirus (SARS-COV) in 2002-2003 and Middle East Respiratory Syndrome coronavirus (MERS-COV), which was recognized as an emerging virus in 2012. Remarkably, these viruses can replicate in the cytoplasm of macrophages, a cell type considered to be a critical innate sentinel for detecting and eliminating invading pathogens. Coronaviruses encode multiple interferon antagonists that likely function to impede and delay the activation of type I interferon (IFN) and interferon stimulated genes (ISGs) and that expression of a constellation of antagonists contributes to pathogenesis. A recent study using SARS-COV infection of mice documented the delayed and limited production of interferon that contributes to disease.

Existing vaccine approaches for coronaviruses diseases are based on spontaneous natural attenuation, virus inactivation, and recombinant viral structural proteins via expression vectors. The existing vaccine candidates do not elicit robust protective immune responses. This lack of long term protection may be due to inefficient induction of innate immune response, such as type 1 interferons, which are critical molecules for promoting adaptive immunity and immune memory.

In view of the above, it can be appreciated that there is an ongoing desire to treat coronaviruses and that it would be desirable if a vaccine were available for inoculating subjects against various coronaviruses, including a vaccine that can stimulate both strong innate immune response and effective adaptive immune protection.

### BRIEF DESCRIPTION OF THE INVENTION

The present invention provides mutant coronaviruses, vaccines comprising mutant coronaviruses, methods of producing vaccines, and methods of preventing disease in subjects.

According to one aspect of the invention, a live, attenuated coronavirus is provided that includes a variant replicase gene encoding polyproteins comprising a non-structural protein (nsp)-15. The replicase gene encodes the nsp15 and causes any change, including mutations and/or deletions, that affects the stability or activity of the nsp15.

Other aspects of the invention include variant replicase genes comprising the above-described coronavirus, proteins encoded by such variant replicase genes, plasmids comprising such variant replicase genes, vaccines comprising the above-noted coronavirus, and methods for treating or preventing a disease in a subject by administering such a vaccine to the subject.

According to another aspect of the invention, a method of preventing a disease in a subject is provided that includes activating type I interferon in the subject, wherein activation of the type I interferon reduces the pathogenicity of a coronavirus.

According to another aspect of the invention, a method of producing a vaccine is provided that includes modifying a wild-type coronavirus to produce a live, attenuated coronavirus comprising a variant replicase gene encoding polyproteins and causing a change, including mutations and/or deletions, in a non-structural protein (nsp)-15 that affects the stability or activity of the nsp15. A vaccine may be produced that includes the coronavirus and a carrier. Administering the vaccine to a subject causes activation of type I interferon in the subject which reduces the pathogenicity of the wild-type coronavirus.

Technical effects of coronavirus as described above preferably include the capability of inoculating a subject with a coronavirus that causes activation of type I interferon, which limits viral replication, dissemination, and disease.

Other aspects and advantages of this invention will be further appreciated from the following detailed description.

### BRIEF DESCRIPTION OF THE DRAWINGS

FIGS. 1A through 1D include a schematic diagram of murine coronavirus genome highlighting nonstructural protein 15 and conserved residue threonine 98. FIG. 1A is a schematic diagram of the mouse hepatitis virus (MHV) A59 (MHV-A59) genome. PLP1/2: papain-like protease 1/2; ADRP, ADP-ribose-1′-monophosphatase; 3CL^{pro}, 3C-like protease; RDRP, RNA-dependent RNA polymerase; Hel, helicase; ExON, 3′→5′ exonuclease; NendoU, Nidovirus uridylate-specific endoribonuclease; 2′OMT, ribose-2′-O-methyltransferase. FIG. 1 B represents the crystal structure of nsp15, wherein the N domain, M domain, C domain are indicated at 90° C. rotation. T98 is shown in relation to L57 (N domain) and the catalytic residues (C domain). Protein Data Base ID: 2GTH. FIG. 1C indicates sites of mutation(s) for each coronavirus genotype and indication of induction of type I interferon in virus-infected bone marrow derived macrophages (BMDMs). FIG. 1D indicates the sequence alignment of nsp15 T98 region from representative strains of coronavirus sub-groups using Clustal W.

FIGS. 2A through 2D indicate that MHV nsp15 mutant virus activates expression of type I interferon and is impaired for replication in BMDMs. FIG. 2A indicates IFN-α11 mRNA levels in BMDMs infected with Wild-Type (WT) MHV or N15m1 by quantitative RT-PCR. Values were normalized to β-actin and analyzed using unpaired T test. FIG. 2B indicates secreted IFNα protein levels in supernatant of infected BMDMs detected by quantitative ELISA. Values were analyzed using 2way-Anova test. FIGS. 2C and 2D indicate growth kinetics of WT and N15m1 in B6 BMDMs and ifnar^{-/-} BMDMs, respectively (MOI of 0.1). Viral titers of supernatants were collected at indicated hpi and titers were determined by plaque assay using 17CI-1 cells. Values were analyzed using unpaired T test. Data are representative of 2-3 independent experiments and presented as Mean±SD.

FIGS. 3A through 3D indicate MHV nsp15 mutant virus induces a rapid apoptotic cell death in BMDMs. FIG. 3A indicates BMDMs were infected with WT, N15m1, or UV-inactivated N15m1 (UV-N15m1) MHV. At 24 hpi, cell viability of viral infected-BMDMs were quantified using CellTiter Glo assay. Values were analyzed using unpaired T test. FIG. 3B indicates BMDMs were infected with WT, or N15m1 MHV and subsequently treated with either DMSO, zVAD (20 μM), Necrostatin-1 (Nec-1) (25 μM), or VX-765 (20 μM). Cell viability was measured at 24 hpi by CellTiter Glo assay. Values were analyzed using unpaired T test. FIG. 3C indicates BMDMs were inoculated with WT or N15m1 MHV and zVAD (20 μM) was added to the media. At 24 hpi, caspase 3/7 activity was determined by a caspase 3/7-Glo activity assay. Values were displayed in relative light units (RLU) and analyzed using 2way-Anova test. FIG. 3D indicates BMDMs were infected with WT or N15m1 MHV and cell lysates were collected at indicated time points. Western blot detection of cleaved-caspase 3 and N protein. All infections are at an MOI of 0.1. Data are representative of 2-3 independent experiments and presented as Mean±SD.

FIGS. 4A through 4D indicate MHV nsp15 mutant virus infection activates host dsRNA sensors. FIG. 4A indicates BMDMs were infected with WT or N15m1 MHV (MOI of 0.1). At 16 hpi, cells were lysed and 40 μg cell lysate was detected for phospho-eIF2α, eIF2α and viral N protein by western blot. Calnexin serves as a loading control. FIG. 4B indicates PKR inhibitor blocks N15m1-induced apoptosis in B6 BMDMs. Cells were infected with WT or N15m1 (MOI of 0.1) and subsequently treated with the PKR inhibitor C16 (1 μM). Cells were collected and evaluated for caspase 3/7 activity at indicated time points. Values were displayed in relative light units (RLU) and analyzed using 2way-Anova test. FIG. 4C indicates an RNA degradation pattern of 300 ng total RNA extracted from infected-BMDMs using a bioanalyzer (MOI of 1). The RNA integrity numbers (RIN) and the positions of 28S, 18S rRNAs, and tRNA are shown to the bottom and the right of image, respectively. FIG. 4D indicates an RNA degradation pattern of RNAs from infected-BMDMs (MOI of 0.1) treated with C16 inhibitor (1 μM) or zVAD (20 μM) at 18 hpi. Data are representative of 2-3 independent experiments.

FIGS. 5A through 5D indicate T98M mutation causes nsp15 protein instability. FIG. 5A indicates BMDMs infected with WT or N15m1 virus at an MOI of 0.1 were lysed at 16 hpi, and viral N protein, nsp15, and β-actin were detected by western blotting. FIG. 5B indicates WT and T98M mutant of nsp15 were expressed and purified from E. coli. Coomassie blue staining shown the purified Sumo-tagged nsp15 and T98M, which were detected by nsp15 antibody using western blotting (bottom). FIG. 5C indicates Differential Scanning Fluorimetry (DSF) thermal shift analysis of nsp15 wild type (black) and nsp15-T98M mutant (red) protein. FIG. 5D indicates a radiolabeled RNA molecule R16.4 was treated over time with WT Nsp15 or T98M in the presence of 5 mM Mn2+. At the indicated time-point, an aliquot of the reaction was analyzed on a denaturing 20% polyacrylamide gel. The sequence of RNA R16.4 is shown above the gel image. The only uridylate, at position 13, is underlined. Data are representative of 2-3 independent experiments.

FIGS. 6A through 6D indicate N15m3 virus (nsp15-H262A) phenocopies N15m1 virus in the loss of interferon antagonism. FIG. 6A indicates B6 BMDMs were infected with WT or nsp15 mutant viruses (N15m1 or N15m3) at an MOI of 0.1. At 12 hpi, total RNA was extracted and analyzed for IFN-α11 mRNA levels. Values were presented as Mean±SD and analyzed using unpaired T test. FIG. 6B indicates B6 or ifnar^{-/-} BMDMs were infected WT or N15m3 at an MOI of 0.1. At indicated time points, cell supernatants were collected for plaque assay in 17CL-1 cells. Values were presented as Mean+SD and analyzed using unpaired T test. FIG. 6C indicates B6 BMDMs were infected with WT or N15m3 at an MOI of 0.1 and harvested for analysis of caspase 3/7 activity at indicated time points. Values were presented as Mean±SD and analyzed using unpaired T test. FIG. 6D indicates RNA degradation pattern of 500 ng total RNA extracted from infected-BMDMs using a bioanalyzer (MOI of 0.1). The RNA integrity numbers (RIN) and the positions of 28S, and 18S rRNAs are pointed to the bottom and the right of image, respectively. Data are representative of 2-3 independent experiments.

FIGS. 7A and 7B indicate mutation of Nsp15 affects dsRNA distribution in virus-infected BMDMs. In FIGS. 7A and 7B, BMDMs were infected with WT or N15m3 at MOI of 0.1. Cells were fixed at 6 hpi and stained with anti-dsRNA, anti-nsp2/3, and Hoescht 33342 (FIG. 7A) or anti-dsRNA, anti-nsp15, and Hoescht 33342 (FIG. 7B). Surfaces for puncta were created based on dsRNA fluorescence, and nsp fluorescence was measured within each surface. The number of foci from 25 images was counted using IMARIS software program. Ratio of dsRNA/nsp2/3 was calculated by dividing total dsRNA foci by the number of nsp2/3 foci (FIG. 7A). Percent co-localization of nsp15 with dsRNA was calculated by dividing dsRNA+ nsp15+ foci by total dsRNA foci. Values were analyzed by unpaired T test. Scale bar: 5 μM.

FIGS. 8A through 8G indicate MHV Nsp15 mutant virus is highly attenuated in mice and induces a protective immune response. FIG. 8A indicates Virus burden in organs from mice infected with WT or N15m1 virus. Six-week old C57BL/6J mice (n=4) were intraperitoneally inoculated with 6.0×104 PFU virus. Liver and spleen were harvested at indicated time points and tested for viral titer by plaque assay. Red dash line is an indication of limit of detection. FIG. 8B indicates C57BL/6J mice were intraperitoneally inoculated with 6.0×104 PFU virus. At 24 hpi, mesenteric lymph nodes (MLN) were harvested and viral genomes were measured by quantitative RT-PCR targeting N gene. Values are normalized to β-actin. ***, p<0.001, unpaired t test. FIG. 8C indicates liver pathology by Hematoxylin and Eosin (H&E) staining as indicated by arrows in WT and N15m1-infected mice. Data are representative of 5 mice. In FIGS. 8D and 8E, mice were inoculated by intracranial (IC) injection with 600 PFU of WT or N15m1 virus. Viral pathogenicity was measured by percent body weight loss (FIG. 8D) and percent survival (FIG. 8E). In FIGS. 8F and 8G, thirteen-week old naïve mice and N15m1-infected mice from FIG. 8D were challenged with 6.0×103 PFU WT virus by intracranial (IC) inoculation. Viral pathogenicity was measured by percent body weight loss (FIG. 8F) and percent survival (FIG. 8G). Mouse numbers (n) are indicated. The p values of survival rate were calculated using log-rank test.

FIGS. 9A and 9B indicate porcine epidemic diarrhea virus (PEDV) EndoU-deficient mutant augments type I interferon production in infected-porcine macrophages. FIG. 9A is a schematic diagram of infectious clones of wild-type PEDV (icPEDV) and a PEDV nsp15 mutant virus (icPEDV-deEndoU). FIG. 9B indicates Vero cells were infected with 0.1 plaque forming unit/cell of either icPEDV or icPEDV-deEndoU. At indicated time points, cell supernatants were collected for viral titration. The data demonstrate that both icPEDV and icPEDV-deEndoU propagated efficiently in Vero cells and exhibited a similar growth kinetics. FIG. 9C indicates primary porcine alveolar macrophages (PAMs) were infected with either icPEDV or icPEDV-deEndoU virus and harvested at indicated time points. Total RNA was extracted from the harvested cells and was transcribed into cDNA. The production of type I interferons (IFN-α and IFN-β) was evaluated by examining their mRNA levels using quantitative PCR. The data demonstrates that the PEDV deEndoU mutant virus stimulates a significant high level of type I IFN production in PAMs compared to wild-type PEDV infection (N.D.=not detected).

FIGS. 10A and 10B indicate N15m1, but not N3m virus, induces IFN activation. FIG. 10A indicates BMDMs were infected with WT or N15m1 MHV (MOI of 0.1). At 12 hpi, infected cells were fixed with 4% formaldehyde and stained with Hoechst 33342 (nucleus; blue), anti-nucleocapsid (N; red), and anti-ISG54 (green). Immunofluorescence was detected by confocal microscopy. FIG. 10B indicates BMDMs were infected with WT, N3m, or N15m1 MHV (MOI of 0.1). IFN-α11 mRNA levels were determined at 12 hpi by quantitative RT-PCR. Values normalized to β-actin. n.s., not significant, unpaired t test.

FIGS. 11A and 11B indicate N15m1 induces a rapid apoptotic cell death. FIG. 11A indicates B6 BMDMs were infected with WT, N15m1, or UV-inactivated N15m1 (UV-N15m1) MHV. At 24 hpi, cytopathic effect was observed under bright-field microscopy. FIG. 11B indicates condensation of nuclear chromatin (black arrows) indicative of apoptosis was determined by electron microscopy in BMDMs treated with staurosporine (1 μM) or infected with WT or N15m1 MHV at 16 hpi.

FIGS. 12A and 12B indicate N15m1-induced apoptosis can be inhibited by PKR inhibitor C16 and requires type I IFN receptors. FIG. 12A indicates B6BMDMs were infected with WT or N15m1 at an MOI of 0.1. Cytopathic effect and cell viability were evaluated at 18 hpi. ****, p<0.0001, unpaired T test. FIG. 12B indicates B6 or ifnar^{-/-} BMDMs were infected with WT or N15m1 at an MOI of Caspase 3/7 activity was measured at 18 hpi by a Caspase 3/7-Glo assay. ****, p<0.0001, 2way-Anova test. Data are representative of 2-3 independent experiments. Data in FIG. 12B are displayed in relative light units (RLU) and presented as Mean±SD.

FIG. 13 indicates T98M mutation alters the oligomerization of nsp15.Dynamic Light Scattering was used to evaluate the percent monomer and hexamer present at increasing concentrations of WT and T98M nsp15.

FIGS. 14A and 14B indicate N15m3 induces a rapid cell death in B6 BMDMs. B6 BMDMs were infected with WT, N15m1, or N15m3 virus at an MOI of 0.1. FIG. 14A indicates at 24 hpi, cell viability was determined using CellTiter Glo assay. Values were analyzed by unpaired T test. FIG. 14B indicates at 12 hpi, cell was harvested and 20 ug total lysate was used for the detection of nsp15, N protein, and loading control (β-actin).

FIGS. 15A and 15B indicate Nsp15 endonuclease activity does not alter the amount of dsRNA in infected BMDMs. B6 (FIG. 15A) or ifnar^{-/-} BMDMs (FIG. 15B) were infected with WT or N15m3 virus at an MOI of 0.1. At 6 hpi, cells were stained for dsRNA and analyzed by flow cytometry.

FIGS. 16A and 16B indicate Nsp15 affects the distribution of dsRNA in infected BMDMs. BMDMs were infected with WT or N15m3 at MOI of 0.1. Cells were fixed at 6 hpi and stained with anti-dsRNA, anti-nsp2/3, and Hoescht 33342. The number of foci from 25 images was counted using IMARIS software program. FIG. 16A indicates the number of dsRNA and nsp2/3 foci were counted in ifnar^{-/-} BMDMs. FIG. 16B indicates the localization of dsRNA and nsp2/3 in B6 BMDMs. The number of dsRNA and nsp2/3 foci were counted and the ratio of dsRNA/nsp2/3 was calculated by dividing total dsRNA foci by the number of nsp2/3 foci (FIG. 16A). Values were analyzed by unpaired T test. Scale bar: 5 μM.

FIGS. 17A and 17B indicate Nsp15 mutant viruses are attenuated in C57BL/6 mice but not in ifnar^{-/-} mice. Six-week old C57BL/6 Mice (n=5) were intraperitoneally inoculated with 6.0×104 PFU virus. At 5 dpi, liver pathology was determined by H&E staining (FIG. 17A). Liver were harvested at 3 and 5 dpi and tested for viral titer by plaque assay using 17CI-1 cells (FIG. 17B). Red dashed line indicates the limit of detection.

### DETAILED DESCRIPTION OF THE INVENTION

The present disclosure provides mutant coronaviruses, vaccines comprising mutant coronaviruses, methods of producing vaccines, and methods of preventing disease.

RNA viruses that replicate via dsRNA intermediates can be detected as “non-self” by host dsRNA sensors, including cytoplasmic RIG-like receptors (RLRs). Activation of RLRs stimulate the production of interferon, which upregulates additional dsRNA sensors, such as PKR and the OAS/RNase L system, and hundreds of antiviral interferon-stimulated genes (ISGs). Additionally, interferon secreted from virus-infected cells that successfully sense the dsRNA can induce an antiviral state in neighboring cells and limit replication of potentially invading RNA viruses. Thus, many viruses have evolved strategies to sequester dsRNA to escape detection by host sensors. This disclosure presents a previously unrecognized role for coronavirus nsp15 in blocking the activation of dsRNA sensors in macrophages, thus enabling viral replication and the dissemination of the progeny virus.

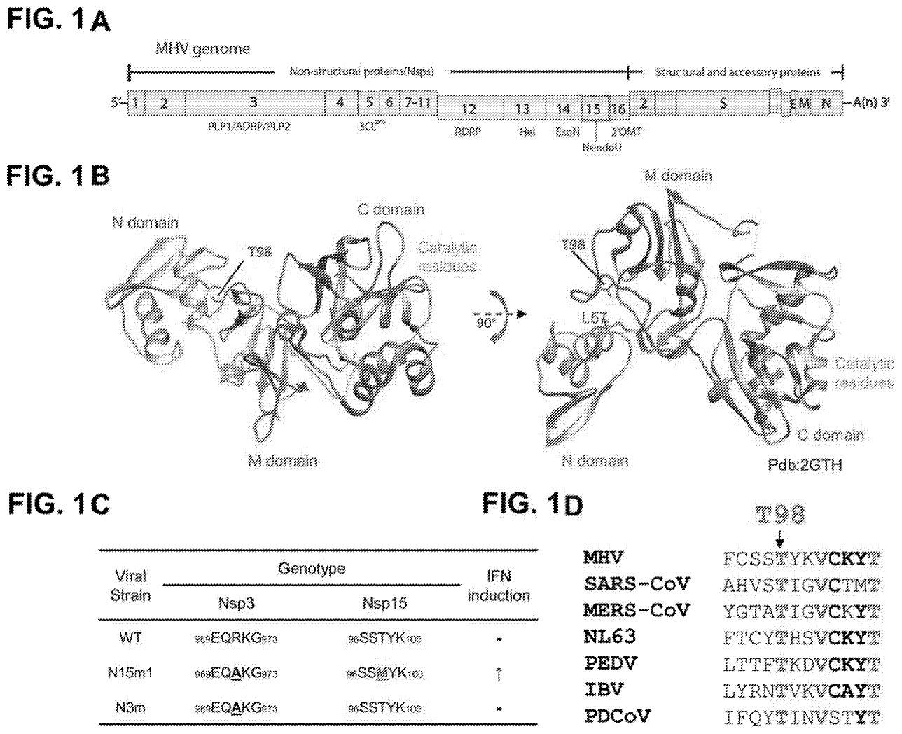

To investigate coronavirus antagonism of the interferon response, investigations leading to the present invention tested mouse hepatitis virus strain A59 (MHV-A59), a model coronavirus that replicates in multiple murine cell types, including macrophages, and can cause acute hepatitis or lethal encephalitis, depending on the site of injection. The viral genomic RNA is thirty-two kilobases and two-third of the genome encodes a large replicase polyprotein, while the remainder of the genome codes for structural proteins and strain-specific accessory proteins (FIG. 1A). The replicase polyprotein is processed by viral proteases into sixteen nonstructural proteins (nsp's). The viral nsp's assemble together with host endoplasmic reticulum to generate convoluted membranes and double membrane vesicles (DMVs), which are the sites of viral RNA synthesis. Coronavirus RNA replication proceeds via the generation of a nested-set of negative-strand RNAs that serve as the templates for synthesis of new positive-strand genomes and mRNAs. Double-stranded RNA (dsRNA) intermediates, potent stimulators of cytoplasmic innate sensors, are produced during this process and visualized in association with the DMVs. A potential function for coronavirus DMVs may be to sequester viral dsRNA away from host dsRNA sensors. However, it is unclear if DMVs alone are sufficient to prevent activation of the host innate immune response. Investigations reported herein surprisingly showed that coronavirus nonstructural protein 15 (nsp15), a highly conserved Nidovirus (coronaviruses and arterivuses) component with endoribonuclease activity, acts in conjunction with the viral replication complex to limit the exposure of viral dsRNA to host dsRNA sensors.

Nsp15 is a nonstructural protein generated by viral protease-mediated processing of the replicase polyprotein (FIG. 1A). Bioinformatic analysis revealed that the nsp15 contains a domain with distant homology to cellular endoribonucleases, termed NendoU, which is highly conserved in vertebrate Nidoviruses. Structural and biochemical studies revealed that SARS-COV and MHV nsp15, and the arterivirus ortholog nsp11 can assemble to form oligomers and cleave ssRNA and dsRNA molecules with a 3′ uridinylate preference. However, the role of endoribonuclease activity in Nidovirus replication and pathogenesis has previously not been well understood. Researchers were unable to recover human CoV 229E virus encoding an endoribonuclease catalytic site mutant, and therefore concluded that nsp15 was essential for coronavirus replication; while MHV encoding nsp15 catalytic site mutations replicated to reduced titers (about one log) in fibroblast cell lines.

Investigations leading to the present invention determined that nsp15 is not required for viral RNA synthesis per se, but acts to mediate evasion of host dsRNA sensors. Specifically, coronaviruses encoding mutations in nsp15, which either renders nsp15 unstable or disables endoribonuclease activity, activate interferon and dsRNA sensors PKR and OAS to promote apoptotic cell death in macrophages. Therefore, nsp15 is essential for virus infection and dissemination in mice, and that a nsp15 mutant virus can induce a protective immune response.

Nonlimiting embodiments of the invention will now be described in reference to experimental investigations leading up to the invention.

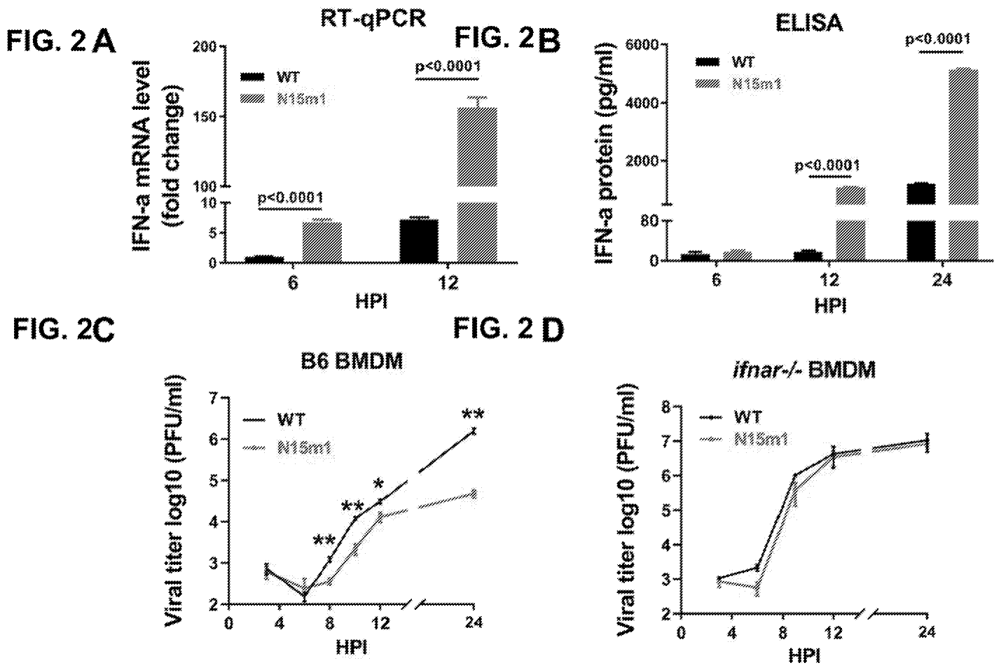

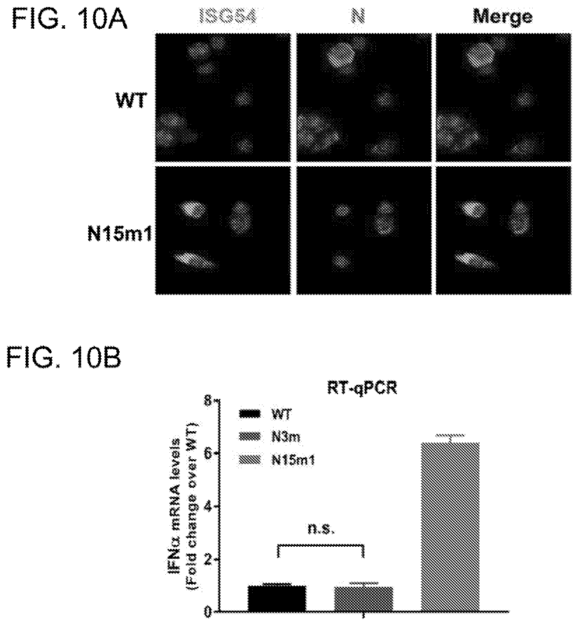

Screening methods were used to identify mechanisms used by coronaviruses to block the innate immune response, particularly the activation of type I interferon (IFN-α/β). These screenings identified a viral isolate, designated N15m1, which elicited production of IFN-α upon infection of murine bone marrow-derived macrophages (BMDMs) (summarized in FIG. 1C). Deep sequencing of viral genomic RNA revealed that N15m1 contains a mutation in nsp15 (threonine 98 to methionine [T98M]) and a mutation in nsp3 (arginine 971to alanine [R971A]). Infection of macrophages with N15m1 activated the transcription of IFN-α as detected by quantitative PCR (qPCR) (FIG. 2A), and increased the amount of secreted IFN-α protein, as detected by ELISA (FIG. 2B). A consequence of type I interferon activation was upregulation of the interferon-stimulated ISG54, as detected by immunofluorescence staining (FIG. 10A). The threonine-98 residue in nsp15 is highly conserved in coronaviruses (FIG. 1D) and may be critical for functionality. In addition, a “clean” nsp3 mutant virus, designated N3m (FIG. 1C), was engineered which harbors only the R971A mutation. N3m retained the ability to antagonize interferon as WT virus (FIG. 10B), further suggesting that the T98M mutation in nsp15 is responsible for the loss of IFN-α antagonism.

To determine whether the expression of IFN-α and ISGs altered virus replication kinetics or titer, wild-type (B6) and type I interferon receptor-deficient (ifnar^{-/-}) BMDMs were infected with either WT or N15m1 virus at a low multiplicity of infection (MOI of 0.1), and monitored virus production over time. The replication of N15m1 was significantly delayed and reduced in B6 BMDMs and the level of progeny virus produced was significantly lower than that of WT virus after 8 hpi (FIG. 2C). This replication deficiency of N15m1 was not observed in ifnar^{-/-} BMDMs as N15m1 had similar kinetics as WT virus (FIG. 2D), consistent with the idea that interferon limits virus replication. Taken together, it was concluded that the T98M mutation in nsp15 results in a virus that fails to block the activation of type I interferon in macrophages. These results indicate that the wild-type nsp15functions as a type I IFN antagonist in the context of viral infection.

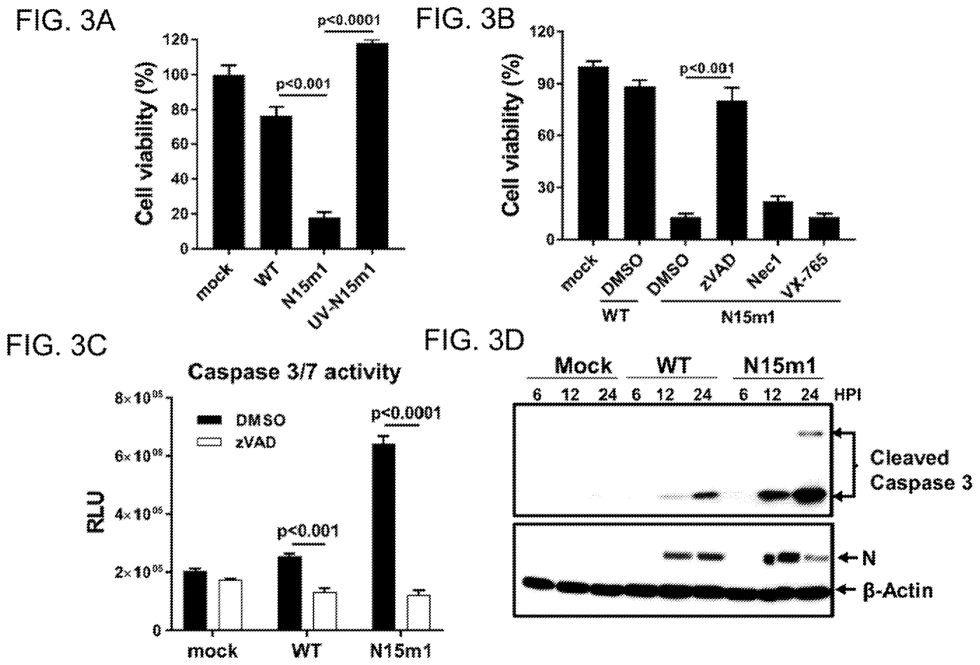

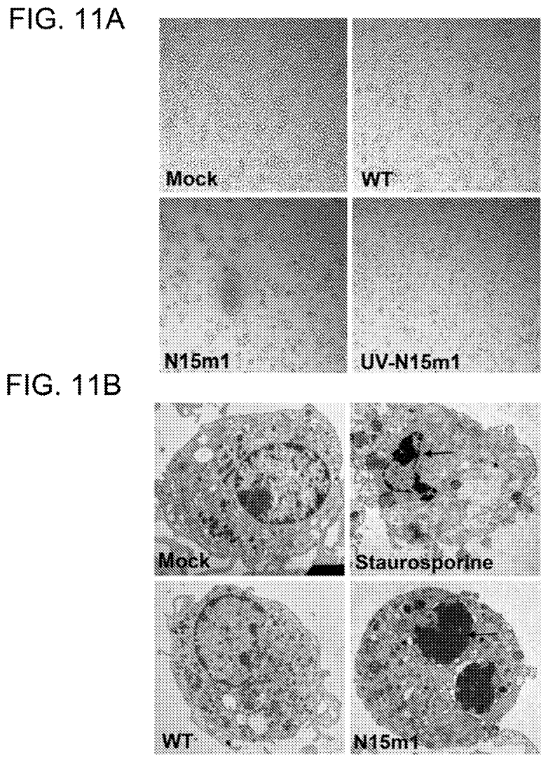

While performing the growth kinetic experiments described above, it was observed that N15m1-infected macrophages died more rapidly than cells infected with either WT virus or UV-inactivated virus. As shown in FIG. 3A, N15m1-infected BMDMs had a significantly lower cell viability at 24 hpi (see also FIG. 11A). In addition, the pan-caspase inhibitor zVAD, but not the RIPK1 inhibitor Nec-1 or caspase-1 inhibitor VX-765, prevented virus-induced cell death (FIG. 3B). These results indicated that N15m1 infection activates apoptotic cell death rather than RIPK1/RIPK3-dependent necroptosis or caspase-1-mediated pyroptosis. This finding was supported by assessing other hallmarks of apoptosis: enhanced caspase-3/7 activity in N15m1-infected BMDMs that is inhibited by zVAD (FIG. 3C); activation of the caspase3/7-dependent apoptosis pathway was also demonstrated through detection of increased levels of cleaved caspase-3 products (FIG. 3D); and condensed, marginalized chromatin, and nuclear fragmentation were observed by electron microcopy (EM) in N15m1-compared to WT-infected BMDMs (FIG. 11B). Taken together, these data demonstrated that N15m1 infection induces apoptosis in macrophages, suggesting that WT nsp15 not only antagonizes activation of type I interferon, but also prevents apoptotic cell death.

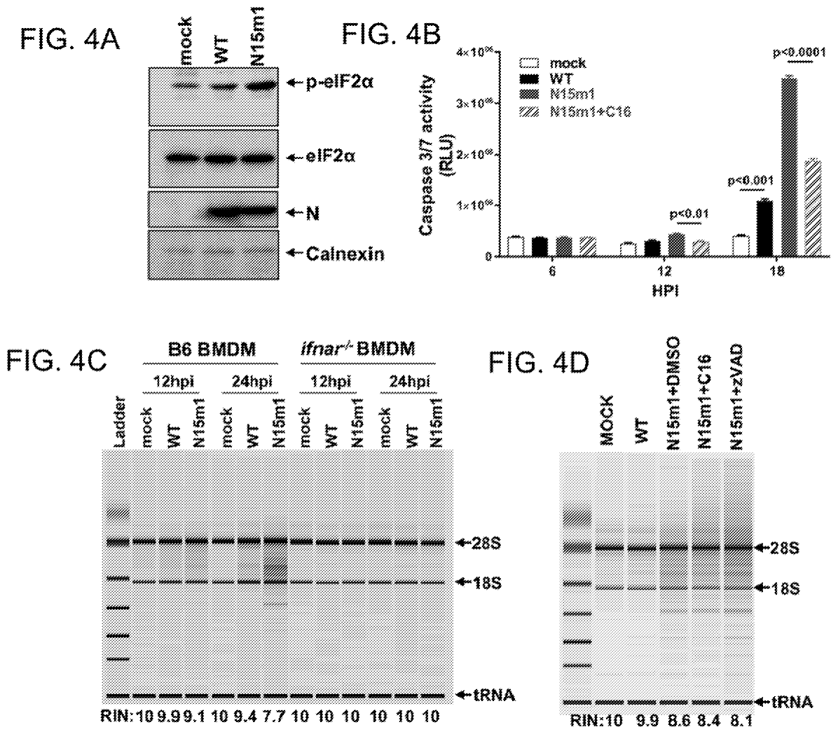

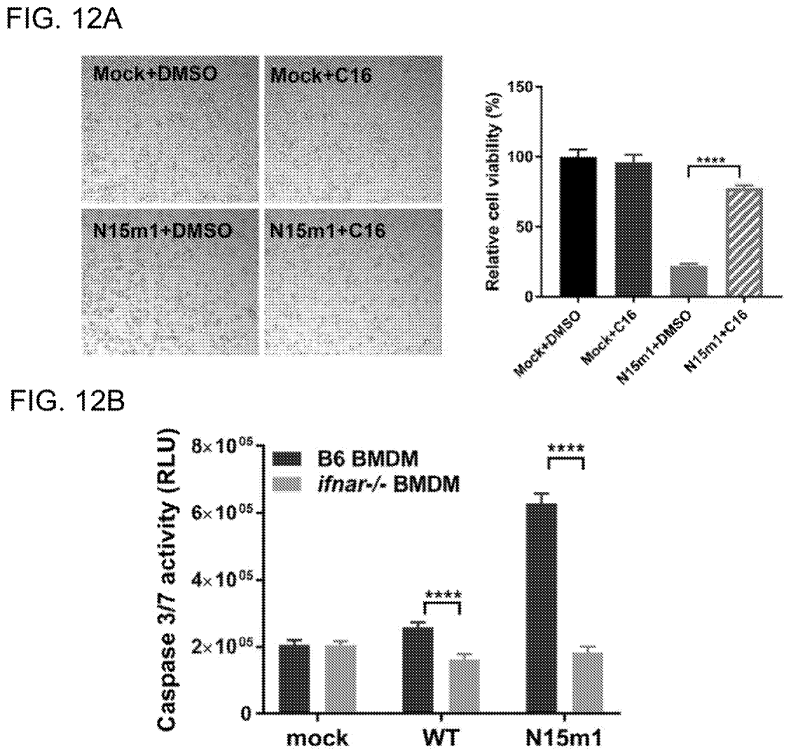

Type I interferon synthesis and apoptotic cell death are triggered by host membrane-associated or cytoplasmic sensors that recognize viral dsRNA. The dsRNA-dependent activation of interferon-stimulated genes 2′5′-oligoadenylate synthetase (OAS) and protein kinase R (PKR) can trigger apoptotic cell death. Previous studies have shown that SARS-COV and MHV nsp15 is a viral endoribonuclease and can both bind to and cleave RNA molecules (ssRNA and dsRNA). Therefore, it was hypothesized that WT nsp15 may block interferon synthesis and prevent apoptosis by sequestering dsRNA from host sensors. To address whether nsp15 prevents the activation of dsRNA sensors, the levels of phosphorylated eIF2α, an indicator of PKR activation, and degradation of ribosomal RNA (rRNA), an indicator of active 2′5′-OAS/RNaseL system signaling were evaluated. Increased phosphorylation of eIF2α was observed in BMDMs infected with N15m1 when compared to infection by WT virus (FIG. 4A). Addition of the specific PKR kinase inhibitor C16 significantly reduced the levels of caspase 3/7 activation that is associated with apoptosis in N15m1-infected cells (FIG. 4B and 12A).

The activation of the 2′5′-OAS/RNaseL pathway was also observed, as revealed by degradation of rRNA at 12 and 24 hpi (FIG. 4C). Degradation of host rRNA was not inhibited by zVAD or C16 (FIG. 4D), indicating that activation of 2′5′-OAS/RNaseL was not a consequence of apoptosis, and was independent of the PKR pathway. Finally, caspase-3 cleavage or activation of caspase-3/7 in N15m1-infected macrophages were dependent on the IFN receptor (FIG. 4C and 12B), further suggesting that the observed apoptosis depends on interferon-stimulated genes. Taken together, these data suggested that during N15m1 infection of macrophages, viral dsRNA detection triggers eIF2α phosphorylation, RNA degradation, activation of caspase 3/7, and apoptosis. These results supported the hypothesis that WT nsp15 functions to prevent dsRNA-mediated activation of innate immune responses.

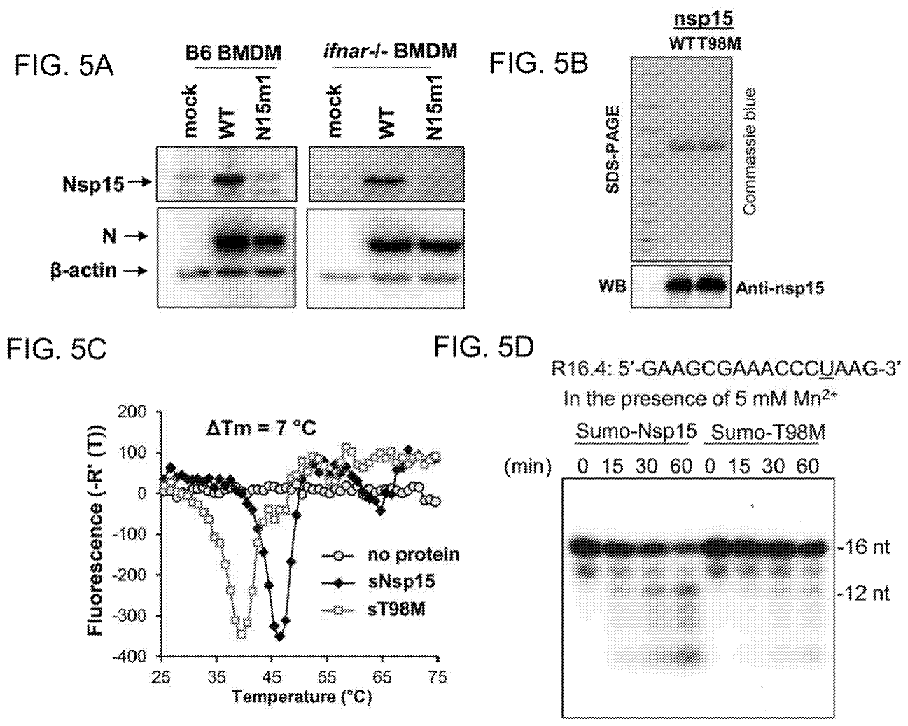

The T98M mutation resides at the interface between the N-terminal domain and middle domain of nsp15 (FIG. 1B), which could affect stability or assembly of oligomers. Indeed, it was found that the level of nsp15 was significantly reduced in N15m1-infected cells when compared to WT-infected cells, while the level of N protein was only minimally reduced (FIG. 5A). The decreased level of nsp15 in N15m1-infected B6 BMDMs was not solely due to the reduction of viral replication because the reduction was also observed in N15m1-infected ifnar^{-/-} BMDMs (FIG. 5A), where both viruses had similar levels of N protein and similar growth kinetics (FIG. 2D). The reduced detection is also not due to the affinity of nsp15 antibodies to mutated protein as both wild-type and T98M nsp15 proteins purified from E. coli were detected equivalently by nsp15 polyclonal antisera (FIG. 5B, bottom). These results suggested that the T98M mutation destabilizes nsp15, resulting in a decrease in the steady-state level of the protein in N15m1-infected cells.

To further evaluate the effect of the T98M mutation on the nsp15 protein, codon-optimized versions of the wild-type and T98M nsp15 proteins were cloned and expressed as SUMO-fusion proteins in E. coli (FIG. 3B, top). Based on the structure of the nsp15, the SUMO tag will not affect the assembly of oligomers, such as hexamers previously reported for nsp15. To further evaluate if the T98M mutation destabilizes the protein, differential scanning fluorimetry (DSF) was used to measure the stability of the nsp15 in response to heat (FIG. 5C). WT nsp15exhibits a major transition to the denatured state at 47° C. In contrast, the T98M mutant denatured at 40° C., seven degrees lower than the WT nsp15, further indicating that the T98M mutation renders nsp15 less stable.

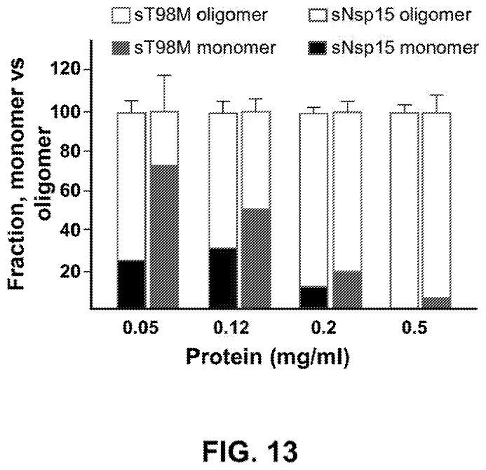

Using dynamic light scatter (DLS) spectrometry, it was found that the majority of WT nsp15 assembles to form oligomers at a protein concentration of 0.05 mg/ml (FIG. 13). In contrast, the major of the nsp15-T98M mutant was detected in the monomeric form under these conditions (FIG. 13). To determine if impaired or lack of oligomerization of nsp15-T98M affected endoribonuclease activity, an evaluation was conducted that found that the T98M mutant exhibited a significant reduction in RNA cleavage activity compared to WT protein (FIG. 5D). Together, the in vitro characterizations of the WT and T98M nsp15 proteins demonstrated that T98M mutation decreases protein stability and impairs endoribonuclease activity. These results were interpreted to further demonstrate that a variant replicase gene encoding polyproteins comprising nsp15 and causing any change, including mutation(s) and/or deletion(s), in nsp15 has the ability to affect the stability (destabilize) or activity (inactivate) of nsp15.

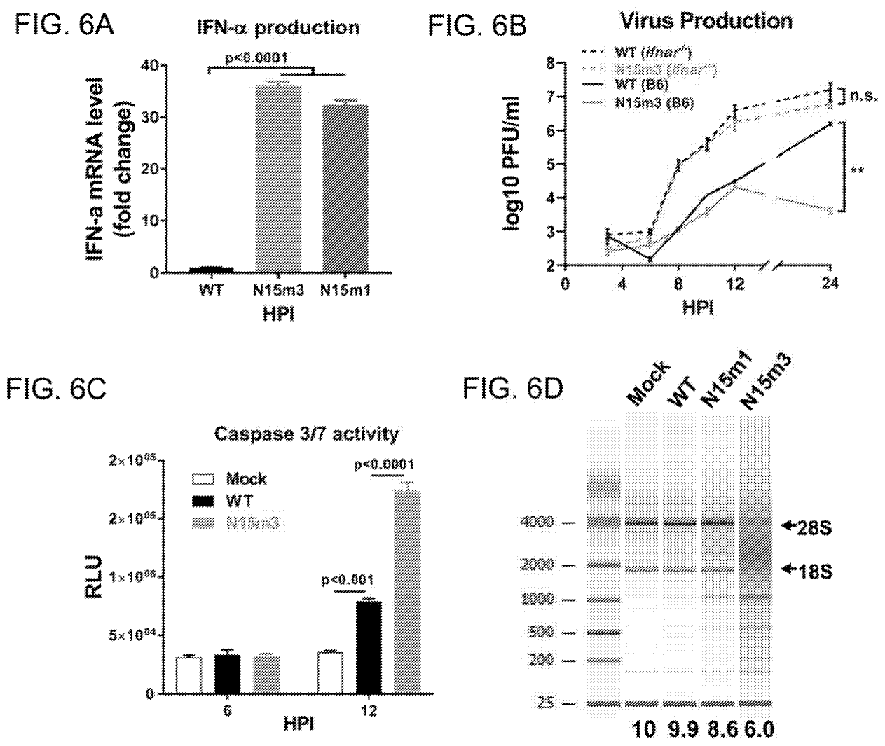

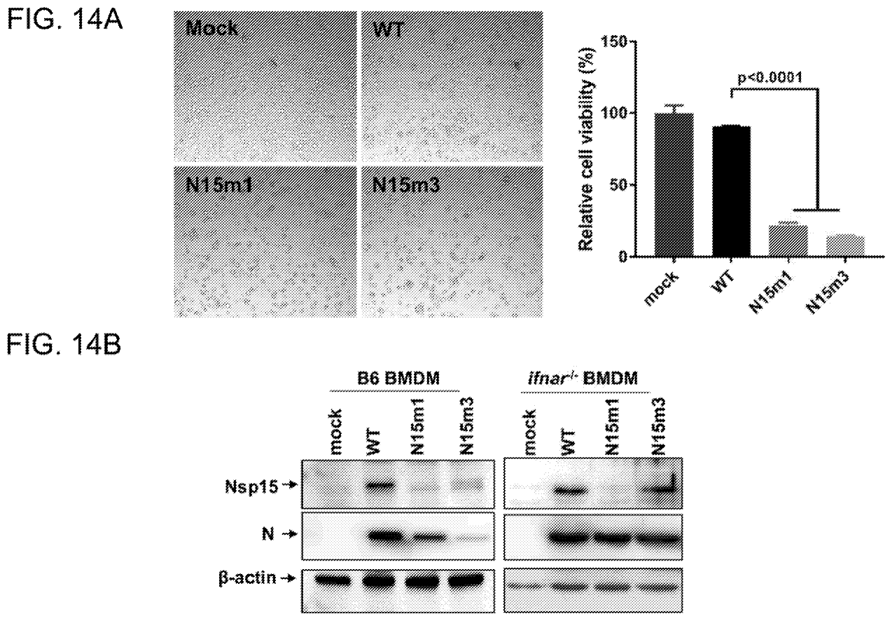

Since T98M mutation causes loss of protein and impairs endoribonuclease activity of nsp15, experiments were performed to determine whether endoribonuclease activity is critical for evasion of host dsRNA sensors. A mutant virus with an endonucleolytic inactive form of nsp15 (H262A), designated as N15m3, was generated. It was found that infection of macrophages with N15m3 resulted in elevated transcription of IFN-α (FIG. 6A), replication deficiency in B6 but not in ifnar^{-/-} BMDMs (FIG. 6B), rapid apoptotic cell death (FIG. 6C and 14A), and rRNA degradation (FIG. 6D), demonstrating that N15m3 phenocopies N15m1 virus. The H262A mutation did not affect the steady-state level of nsp15 since N15m3, unlike N15m1, expressed similar levels of nsp15 as WT virus in ifnar^{-/-} BMDMs (FIG. 14B). Thus, it was determined that the loss of endoribonuclease activity (H262A) recapitulates the phenotypes associated with the loss of nsp15 protein by T98M mutation. Overall, these results indicated that nsp15 endoribonuclease activity is important for evasion of host dsRNA sensors.

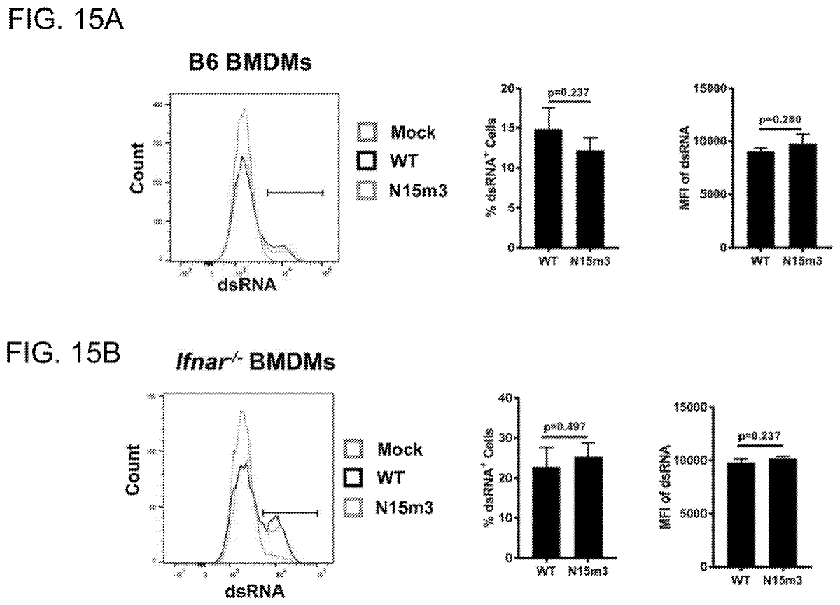

Purified MHV nsp15 from E. coli exhibits endonuclease activity in the presence of manganese and the substitution of the catalytic Histidine 262 with Alanine (H262A) resulted in an inactive enzyme. Therefore, the N15m3 virus was chosen as it harbors the catalytic inactive form of nsp15 (H262A) to further determine the role of endoribonuclease activity in preventing the activation of host dsRNA sensors. As CoV infection produces dsRNA intermediates during virus replication, it was hypothesized that nsp15 may degrade viral dsRNA to prevent the accumulation of dsRNA. To test this hypothesis, either B6 or ifnar^{-/-} BMDMs were infected with WT or N15m3 virus and the level of dsRNA was measured. Surprisingly, an increased level of dsRNA in N15m3-infected cells as measured by the fluorescence intensity of dsRNA or the percentage of dsRNA positive cells using flow cytometry (FIG. 15) was not observed, implying that the antagonistic function of nsp15 may not be mediated through degradation of viral dsRNA.

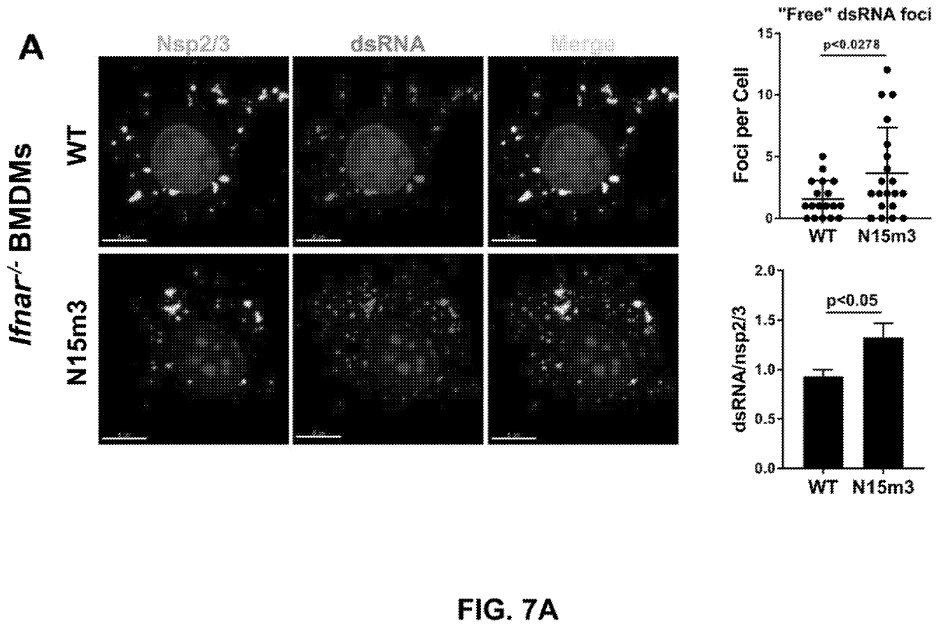

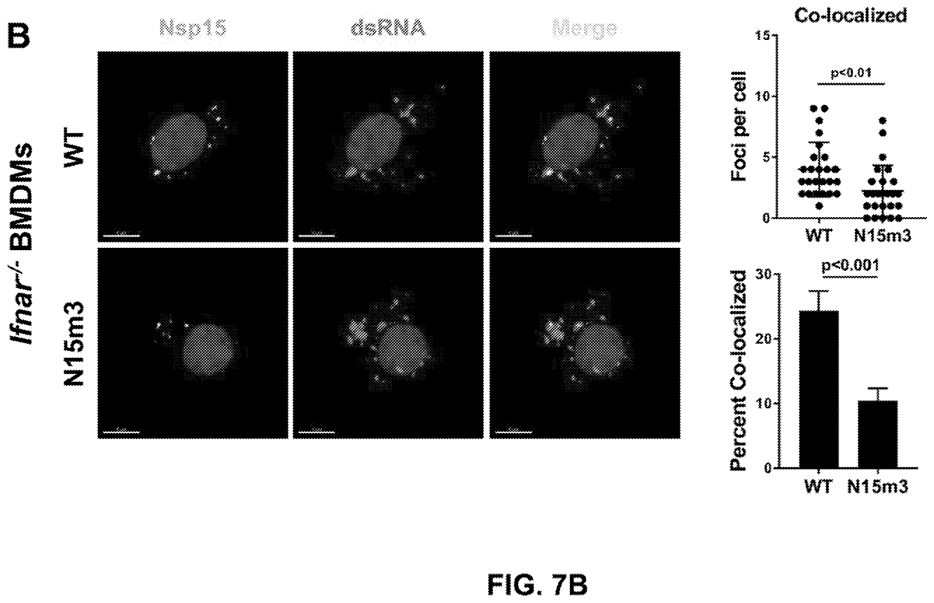

It was believed that CoV dsRNA mainly associates with replication complex and is buried in DMVs, which are thought to protect viral RNA from host sensors. Therefore, it was hypothesized that nsp15 may function to maintain the association of dsRNA with the replication complex or to facilitate dsRNA packing into DMVs. Thus, it was predicted that the nsp15 mutant virus may generate more “free” dsRNAs, and that these free dsRNAs activate host sensors. To test this hypothesis, the subcellular localization of dsRNA and replication complex (nsp2/3 as an indicator) was evaluated by immunofluorescence using specific antibodies. Interestingly, it was found that N15m3 infection yielded more dsRNA foci that did not co-localize with nsp2/3, particularly in ifnar^{-/-} BMDMs (FIG. 7A).

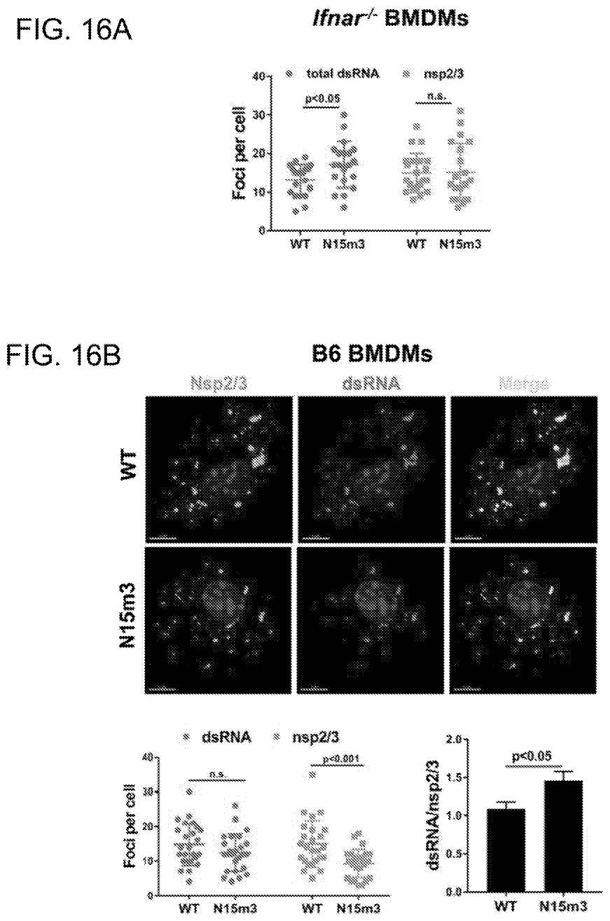

To quantify the “free” dsRNA, the number of foci of dsRNA and nsp2/3was counted using the IMARIS software program. It was found that in the case of having similar numbers of nsp2/3 foci in ifnar^{-/-} BMDMs, N15m3 produced more dsRNA foci than the WT virus (FIG. 7A left panel and 16A). The number of “free” dsRNA foci and the ratio of dsRNA/nsp2/3 of N15m3 were significantly higher than that of the WT virus (FIG. 7A right panel). In B6 BMDMs, N15m3 virus produced similar numbers of dsRNA foci but significantly less nsp2/3 foci due to the impaired replication (FIG. 6B). It was observed that the ratio of dsRNA/nsp2/3 was still higher in N15m3 than in WT virus (FIG. 16B), consistent with the results obtained from ifnar^{-/-} BMDMs (FIG. 7A). Taken together, these data suggest that nsp15 may not affect the amount of dsRNA in the cell, but does function to maintain the association of dsRNA with nsp2/3, and later dsRNA packing into DMVs.

To further understand the relationship between nsp15 and dsRNA, localizations of nsp15 and dsRNA were examined. It was believed that nsp15 associates with newly synthesized viral RNA in characteristic puncta that contain viral replicase proteins, and are considered to be the sites for viral RNA synthesis. Nsp15 and dsRNA were visualized by immunofluorescence using specific antibodies. It was found that the number of dsRNA foci that co-localized with nsp15 was significantly reduced in N15m3-infected cells as compared to WT infected cells (FIG. 7B). These results further supported that nsp15 may associate with dsRNA and the viral replication complex. Therefore, it was concluded that loss of endoribonuclease activity may disrupt the association of dsRNA and replication complex, resulting in more “free” dsRNA to be sensed by host sensors.

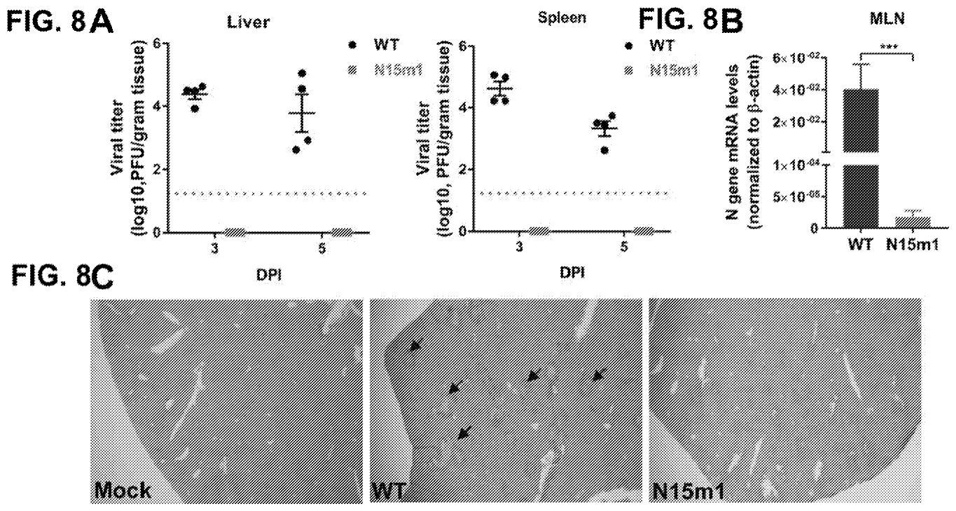

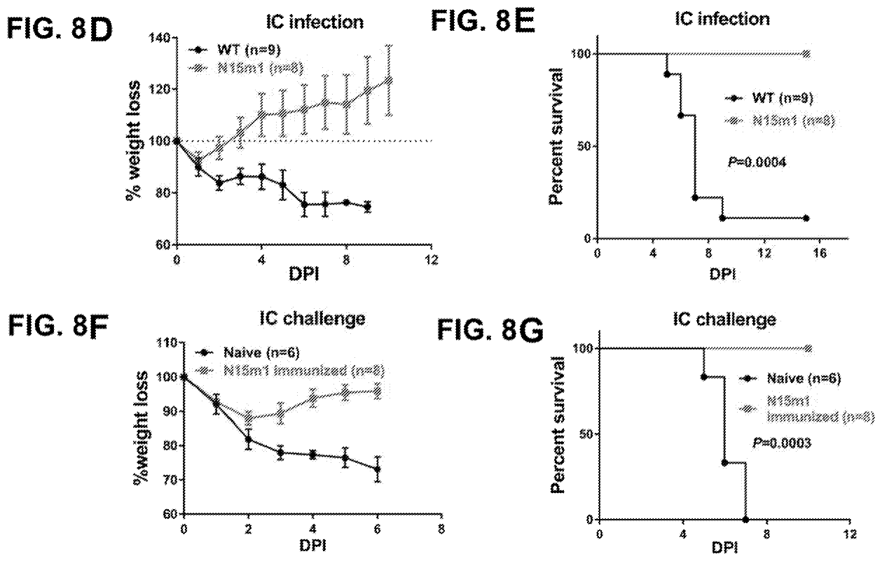

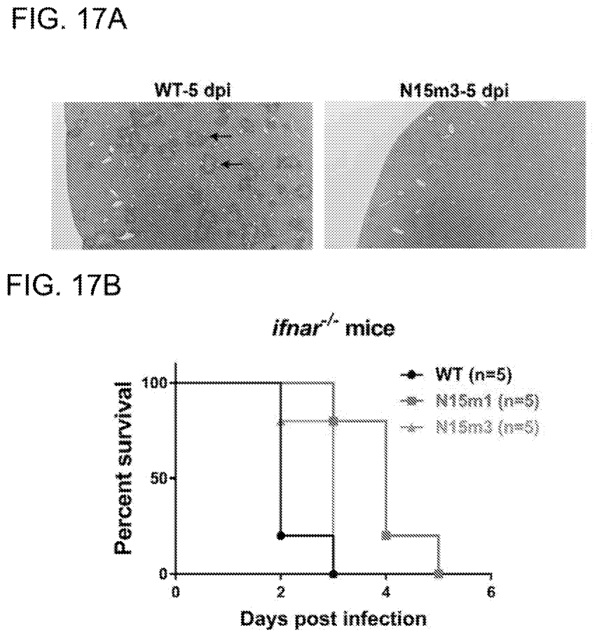

Since nsp15 mutant viruses induce a robust interferon response and activate host dsRNA sensors, additional investigations were performed to determine if the loss of nsp15-mediated antagonism of innate immune responses alters the pathogenesis of murine coronavirus. To this end, a non-lethal model of infection was employed in which WT MHV-A59 titers were expected to peak at day five post-infection. Mice were injected intraperitoneally with 60,000 PFU and the livers and spleens were harvested at days three and five post-infection to measure viral replication. Interestingly, a standard plaque assay to enumerate infectious particles was negative for virus in mice inoculated with N15m1 (FIG. 8A). The more sensitive RT-qPCR assay to detect MHV N gene mRNA identified minimal levels of N15m1 RNA in the mesenteric lymph node (MLN) at day one post-infection but not at later times (FIG. 8B and data not shown). Histological examination of the livers revealed typical lesions associated with infection by WT MHV, but not in N15m1-infected mice (FIG. 8C). Similarly, N15m3-infected mice showed no signs of liver pathology similar to N15m1-infected mice (FIG. 17A).

A lethal challenge model was used to determine whether the N15m1 virus was attenuated. Only 600 PFU of WT virus introduced into a mouse cranium is needed to induce lethal encephalitis. The WT-infected mice lost body weight and succumbed to infection by day seven post-infection. In contrast, all N15m1-infected mice survived the infection, exhibiting only transient weight loss at day one post-infection, and gained weight over time (FIG. 8D & 8E). These data reveal that N15m1 is profoundly attenuated and exhibits no pathogenesis even in this sensitive in vivo model. Moreover, ifnar'-mice were intraperitoneally infected with 50 PFU of either WT or mutant viruses, and all mice rapidly succumbed to infection, supporting the concept that the loss of IFN antagonism function of nsp15 in the mutant viruses is responsible for attenuation during infection of B6 mice (FIG. 17B). Importantly, inoculation with N15m1 fully protected mice from a challenge with 6000 PFU (10-fold lethal dose) of WT MHV (FIG. 8F & 8G).

Overall, these experimental investigations demonstrated the role of nsp15 during WT virus infection in preventing viral dsRNA from activating host sensors. Loss of nsp15 during viral replication resulted in rapid sensing of dsRNA, activation of type I interferon, 2′5′-OAS/RNaseL and PKR, which limits viral replication, dissemination and disease. It was shown that in the absence of IFN signaling, nsp15 mutant viruses replicate similarly as WT virus, indicating that nsp15 endoribonuclease activity is not required for viral RNA synthesis. Instead, nsp15 is critical for derailing the innate immune response to coronavirus replication in macrophages.

The effect of nsp15 as an antagonist of dsRNA sensors is distinct from previous studies of coronavirus replicase-encoded antagonists. CoVs encode a 2′O-methyltransferase (nsp16) to provide a cap structure at the 5′ ends of their mRNA to evade host PRRs recognition. SARS-COV and MHV mutant viruses that lack 2′O-methyltransferase activity triggered the type I IFN response and were attenuated in macrophages and in mice. SARS-COV and MHV nsp1 can prevent IFN induction by degrading the host mRNA; however, infection of macrophages with an nsp1 deletion mutant did not alter IFN induction, but rather, was attenuated in B6 mice. CoVs encode an essential papain-like protease (PLP2 or PLpro) domain within nsp3, which can dysregulate innate immune response by cleaving ubiquitin molecules from cellular substrates. Since nsp15 is highly conserved in all coronaviruses, including relatively benign coronaviruses such as HCoV-229E and OC43, which cause common colds, it is possible that nsp15 is fundamental to the ability of the virus to prevent the recognition of host dsRNA sensors in macrophages allowing for dissemination of the virus to target organs.

Previous studies have shown that coronaviruses encode viral factors to impede the dsRNA-mediated activation of host sensors, such as OAS/RNase L system and PKR. For example, the strain of murine coronavirus used in the above noted investigations also encodes accessary protein ns2, which is another important antagonist of the OAS/RNase L system. Ns2 encodes a 2′,5′-phosphodiesterase (PDE) enzyme, which cleaves 2′,5′-oligoadenylate, the product of OAS, to prevent RNase L-mediated rRNA degradation in macrophages. Interestingly, coronavirus ns2 confers a liver-specific effect as intracranial infection with the ns2 mutant virus remains lethal. It was found that the nsp15 mutant viruses with an intact ns2 were able to induce rRNA degradation in macrophages and were highly attenuated in both hepatitis and encephalitis mouse models. Investigations of the accessory proteins of MERS-COV revealed that NS4b has a similar PDE activity as MHV ns2 and inhibits RNase L activity. MERS-COV NS4a encodes a dsRNA-binding protein that limits the activation of PKR. Deletion of NS4a in the context of the virus was not sufficient to activate PKR and the generation of a stress response, and therefore, MERS-COV must have redundant mechanisms to suppress recognition and activation of dsRNA sensors. The results presented here indicate that nsp15 may serve as a conserved viral factor to suppress the activation of dsRNA sensors during CoV infection, and that additional suppression of PKR and/or OAS/RNase L by ns2, NS4a, or NS4b may provide tissue-specific or redundant mechanisms of control that likely contribute to viral pathogenesis.

The exact mechanism(s) nsp15 uses to suppress the activation of viral RNA sensors remains to be determined, particularly concerning the role of the nsp15 endoribonuclease activity in the antagonism of dsRNA sensors. Regarding ribonuclease activity, two viruses encoding ribonuclease activity have been shown to function in suppressing innate immune responses. The pestivirus RNase Erns acts as an enzymatically active decoy receptor that degrades viral RNA in endolysosomal compartments, which limits the exposure of the RNA to host dsRNA sensors. The nucleoprotein from Lassa virus also contains a 3′ to 5′ exonuclease domain that digests free dsRNA and is essential for suppressing the translocation of interferon regulatory factor three and activation of the host innate immune response system. Thus, it was reasonable to hypothesize that nsp15 may degrade viral dsRNA to prevent the detection by host dsRNA sensors. Nonetheless, increased dsRNA levels were not observed in macrophages infected with nsp15 mutant virus (FIG. 14), suggesting that nsp15 might have specific targets instead of broadly degrading RNA. These investigations showed that nsp15 may maintain the association of dsRNA with the replication complex or mediate DMV packing of dsRNA.

In view of the above, the above-noted investigations provided an understanding the mechanisms used by coronaviruses to effectively modulate the innate immune response in macrophages and provide several new directions for development of therapies targeting nsp15 and development of live-attenuated vaccines. For example, it is believed that subjects may be inoculated against various coronaviruses by application of a vaccine comprising a live-attenuated coronavirus comprising a variant replicase gene encoding polyproteins and causing a change, including mutation(s) or deletion(s), in nsp15 that affects the stability or activity of nsp15, as a particular but nonlimiting example, a protein comprising an amino acid mutation of threonine to methionine at position 98 or catalytic histidine to alanine at position 262. The vaccine may include a pharmaceutically acceptable carrier such as but not limited to water, saline, buffered saline, phosphate buffer, alcohol/aqueous solutions, emulsions or suspensions.

Since nsp15 is highly conserved in all coronaviruses, it is believed that the above-described approach can be extended to generate vaccines for all existing and emerging coronaviruses (Coronavirinae). Therefore, it is believed vaccines can be successfully produced by forming mutations in nsp-15 of various coronaviruses including but not limited to Severe Acute Respiratory Syndrome coronavirus (SARS-COV), Middle East respiratory syndrome coronavirus (MERS-COV), human coronaviruses 229E (HCoV-229E), OC43 (HCoV-OC43), HKU1 (HCoV-HKU1), and NL63 (HCoV-NL63), feline infectious peritonitis virus (FIPV), canine coronavirus (CCOV), infectious bronchitis virus (IBV) of chickens, bovine coronavirus (BoCoV), and porcine coronaviruses including transmissible gastroenteritis virus (TGEV), porcine delta coronavirus (PDCoV), porcine epidemic diarrhea virus (PEDV), porcine respiratory coronavirus (PRCV), and porcine hemagglutinating encephalomyelitis coronavirus (PHE-CoV).

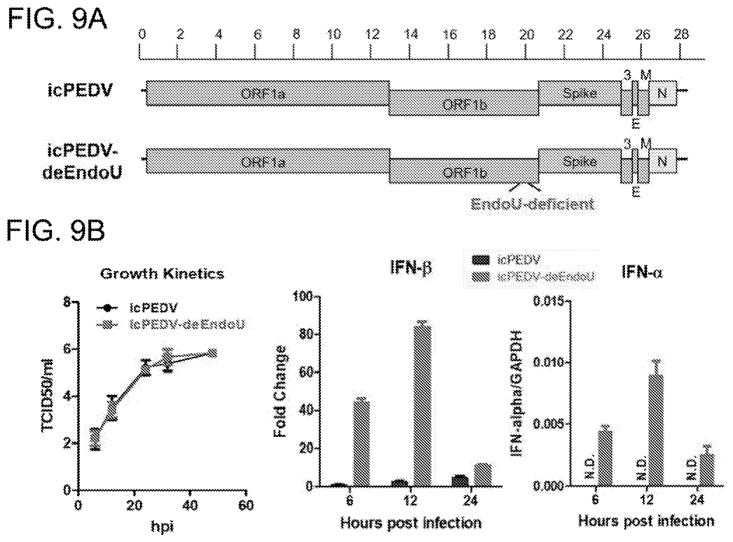

The inactivation of nsp15/EndoU of porcine epidemic diarrhea virus (PEDV) by mutation of an infectious clone results in a virus that replicates efficiently in tissue culture but activates the innate immune response to generate interferons in infected-macrophages (FIG. 9). The inactivation of nsp15/EndoU activity in any coronavirus that infects humans or animals will generate a vaccine strain of the virus. This inactivation allows for rapid activation of interferon in virus-infected macrophages, which can then stimulate the adaptive immune response. This can be demonstrated in multiple species of coronaviruses and results in a virus that stimulates the activation of the innate immune response, for example, the Type I interferon response.

Corroboration of aspects of the study and conclusions discussed above have been published in Deng et al., “Coronavirus nonstructural protein 15 mediates evasion of dsRNA sensors and limits apoptosis in macrophages,” Proceedings of the National Academy of Sciences May 2017, 114 (21) E4251-E4260; DOI: 10.1073/pnas.1618310114, and in Kindler et al., “Early endonuclease-mediated evasion of RNA sensing ensures efficient coronavirus replication,” PLOS Pathog 13(2):e1006195. doi: 10.1371/journal. ppat. 1006195 (2017). The entire contents of these two papers are incorporated herein by reference.

Additional details relating to the investigations described herein are provided below as discussions of experimental procedures used during the investigations. Such discussions are not intended to set forth limitations to the scope of the invention, but instead are provided to disclose the scope and particular details of the investigations.

The following discussion describes certain experimental procedures relating to FIGS. 1A through 8G.

Cells, antibodies, and chemicals. Delayed brain tumor (DBT) cells were grown in MEM supplemented with 10% tryptose phosphate broth, 5% heat-inactivated fetal calf serum (FCS), 2% penicillin/streptomycin, and 2% glutamine. BHK-21 cells expressing the MHV receptor (BHK-R) were cultured in DMEM) supplemented with 10% FCS and G418 (0.8 mg/ml) (SV30069, HyClone). The 17CI-1 cell line was maintained in 5% FCS DMEM. Rabbit anti-nsp2/3 serum (anti-D3) and anti-nsp15 serum (anti-D23). Mouse anti-nucleocapsid (J3.3) was from the University of Wisconsin, Madison. Antibodies purchased commercially: dsRNA (K1, Scicons), ISG54 (PA3845, ThermoFisher), eIF2α (sc-133132) and p-eIF2α (sc-12412) were from Santa Cruz. Chemical inhibitors were from the following sources: pan-caspase inhibitor zVAD (627610, Millipore), Necrostatin-1 (Nec-1) (480065, Millipore), PKR inhibitor C16 (527450, Millipore), staurosporine (ALX-380-014, Enzo Life Sciences) and VX-765 (F7120, UBPbio).

Viruses and deep sequencing. WT MHV strain A59 (GenBank accession #AY910861) was generated by reverse genetics. To generate MHV mutant viruses, nucleotide changes were incorporated into the cDNA fragments of MHV-A59 genome through PCR mutagenesis (primers available upon request). Subsequent generation of virus by reverse genetics was performed. Rescued viruses were plaque-purified, propagated on BHK-R cells, and titrated on 17CI-1cells. Mutant viruses were maintained exclusively in BHK-R cells. All virus stock preparations and plaque-purified isolates used in this study were full-genome deep sequenced (Kansas State University diagnostic laboratory).

Infection and mouse experiments. BMDMs in 12- or 24-well plates were infected with indicated viral strains at an MOI of 0.1 or 1 in serum-free media. For growth kinetics analysis, cell culture supernatants were collected at indicated time points and titrated by plaque assay on 17CI-1 cells. For mouse infection, all experiments were performed using protocols reviewed and approved by the Loyola University Chicago IACUC. For intracranial (i.e.) infections, six-week-old C57BL/6J female mice (Jackson Laboratory) were inoculated with 600 PFU virus and monitored for body weight daily and euthanized when weight loss was over 25% according to the IACUC protocol. For intraperitoneal (i.p.) infection, six-week-old mice were injected with 60,000 PFU and organs were collected at indicated time points. Evidence of liver pathology was determined by H&E staining.

Cell death assays. Cell viability and Caspase 3/7 activity were measured using CellTiter Glo (G7571, Promega) or CaspaseGlo 3/7 (G8091, Promega) respectively, according to the manufacturer's protocol, with modification.

Differential scanning fluorimetry (DSF) assay. DSF was carried out in the Stratagene MX3005P real-time PCR machine. The samples contain 1×SYPRO Orange, 10 μM of the recombinant protein. All samples were heated at a rate of 0.5° C./min, and the fluorescence intensity and Tm (melting temperature) were determined.

RNA cleavage assay. The standard RNA cleavage assay used 1×104 CPM of 5′-end radiolabeled RNA substrate (1 μM final RNA concentration) and 0.026 μM Nsp15 in 50 mM Tris-HCl (pH 7.5), 50 mM KCl, 1 mM dithiothreitol, 5 mM MnCl2 at 30° C. The endoribonuclease reactions were terminated by the addition of a gel-loading buffer that contained 7.5 M urea. Products were separated by electrophoresis in 20% polyacrylamide gels containing 7.5 M urea. Gels were wrapped in plastic and exposed to a Phosphorlmager screen for quantification using Molecular Dynamics software.

The following discussion describes certain experimental procedures relating to FIGS. 10A through 17B.

Cells, antibodies, and other reagents. Delayed brain tumor (DBT) cells were grown in minimal essential medium (MEM) (61100-061, Gibco) supplemented with 10% tryptose phosphate broth, 5% heat-inactivated fetal calf serum (FCS), 2% penicillin/streptomycin, and 2% glutamine. Baby hamster kidney 21 cells expressing the MHV receptor (BHK-R) were cultured in Dulbecco's modified Eagle medium (DMEM) (12100-046, Gibco) supplemented with 10% heat-inactivated FCS and G418 (0.8 mg/ml) (SV30069, HyClone) to maintain selection for MHV receptor expression. The 17CI-1 cell line was maintained in 5% FCS DMEM. WT MHV strain A59 (GenBank accession #AY910861) was generated by reverse genetics and full-genome sequenced. Differentiated (see below) bone marrow-derived macrophages (BMDMs) were maintained in bone marrow macrophage (BMM) media containing DMEM (10-017-CV, Corning) supplemented with 30% L929 cell supernatant, 20% FCS, 1% L-glutamine, 1% sodium pyruvate, and 1% penicillin/streptomycin. Rabbit anti-nsp2/3 serum (anti-D3) and anti-nsp15 serum (anti-D23). Mouse anti-nucleocapsid (J3.3) was from the University of Wisconsin, Madison. Antibodies purchased commercially: mouse anti-βactin (A00702, Genscript), mouse anti-calnexin (610523, BD), donkey anti-rabbit-HRP (711-035-152, Jackson ImmunoResearch), goat anti-mouse-HRP (1010-05, SouthernBiotech), dsRNA (K1, Scicons), ISG54 (PA3845, ThermoFisher), eIF2α (sc-133132) and p-eIF2α (sc-12412) were from Santa Cruz. Chemical inhibitors were from the following sources: pan-caspase inhibitor zVAD (627610, Millipore), Necrostatin-1 (Nec-1) (480065, Millipore), PKR inhibitor C16 (527450, Millipore), and VX-765 (F7120, UBPbio).

Generation of bone marrow-derived macrophage. Bone marrow was collected from femurs of C57BL/6J (000664, Jackson Labs) or ifnar^{-/-} mice obtained from Washington University in St. Louis. 5×10⁶ bone marrow cells were plated in 100×26 mm petri dishes (25387-030, VWR) with 15 mL BMM media with 50 μM β-mercaptoethanol. After 3 days of incubation at 37° C./5% CO₂, 10 mL of BMM media was added. Following another 3 days of differentiation, BMDMs were washed in cold 1×PBS, incubated for 30 min at 4° C. in 10 mL 1×PBS then gently rinsed from the plates by manual pipetting. 1×10⁷ cells/mL were suspended in BMM media with 10% DMSO and stored in liquid nitrogen liquid phase until use. For preparation of L929 cell supernatant, 3.75×10⁵ L929 cells were plated in 75 mL medium (DMEM [10-017-CV, Corning], 10% FCS, 1% L-glutamine, 1% sodium pyruvate, 1% nonessential amino acids, and 1% penicillin/streptomycin) in a T150 flask (10-126-34, Thermofisher). After 6 days of incubation at 37° C./5% CO₂, the supernatant was harvested, filtered, and stored at −20° C. until use. For viral infections, BMDMs were thawed and plated in 100×26mm petri dishes in BMM media without β-mercaptoethanol. After 3 days of incubation at 37° C./5% CO₂, cells were plated onto tissue culture dishes for subsequent infection experiments after 24-hour incubation.

Mutant viruses and deep sequencing. To generate MHV mutant viruses, nucleotide changes were incorporated into the MHV-A59 genome through PCR mutagenesis (primers available upon request) of cDNA fragments. Subsequent generation of virus by reverse genetics was performed. Viral genomic RNA from in vitro transcription (mMESSAGE mMACHINE T7 Transcription Kit; AM1344,Invitrogen Ambion) of ligated cDNA fragments was electroporated into BHK-R cells. Cell supernatant was collected as viral stock following observation of cytopathic effects. Infectious clones were plaque-purified, propagated on BHK-R cells, and titrated on 17CI-1 cells. Mutant viruses were maintained exclusively in BHK-R cells, which do not produce or respond to interferon. All virus stock preparations and plaque-purified isolates used in this study were full-genome deep sequenced (Kansas State University diagnostic laboratory). Briefly, viral RNA was extracted from virus stocks using QIAamp MinElute Virus Spin Kit (57704, QIAGEN), used to generate a cDNA Library and sequenced by Miseq or lon Torrent technology. Mutant MHV sequences were aligned to the wild-type MHV-A59 synthetic construct (GenBank accession #AY910861).

Evaluating viral replication by plaque assay. To determine virus growth kinetics, BMDMs in 24-wells plates were infected with indicated viral strains at an MOI of 0.1 in serum-free media. After 1 h incubation, inoculum was replaced with fresh, complete medium. Cell culture supernatants were collected at indicated time points and titrated by plaque assay on 17CI-1 cells. To determine the viral titer in organs, a portion of tissue was homogenized with 1.0 mm dia. zirconia/silicon beads (11079110z, BioSpec Products) using an automated homogenizer (6.0 m/sec, 40 sec duration) (MP Biomedicals) in serum-free DMEM. The homogenized organs were centrifuged at 10,000×g for 5 min and the clarified supernatants were titrated for viral plaques on 17CI-1 cells. Titers were obtained from three independent assays for each sample. Graphs of virus kinetics were generated using Prism 7 software (GraphPad Software, Inc.).

Cell death assays. Cell viability and Caspase 3/7 activity were measured using CellTiter Glo (G7571, Promega) or CaspaseGlo 3/7 (G8091, Promega) respectively, according to the manufacturer's protocol, with modification. Briefly, 3.0×10⁵ BMDMs/well wereplated in 24-well plates and infected at an MOI of 0.1 for 1 h in serum-free media. Media was replaced with complete media, or for addition of chemical inhibitors, viral inoculum was replaced with completed BMM media containing indicated concentration of inhibitors. Infected BMDMs were incubated for 24 h at 37° C./5% CO₂. BMDMs were washed twice with 1×PBS and lysed with 100 μL Glo reagent/medium mixture (1:1). 50 μL of cell lysate was used for measuring the luminescence signal. Standard deviation and unpaired t test were performed on technical triplicates and data are representative of three independent experiments. Graphs of cell viability were generated using Prism 7 software (GraphPad Software, Inc.).

Immunofluorescence. BMDMs (1.5×10⁵ cell/well) were plated on a glass coverslip in a 24-well plate for 24 h and subsequently infected with wild-type or mutant virus at an MOI of 0.1 in serum-free media. At 6 or 12 hpi, the infected cells were fixed with 4% formaldehyde in 0.095 M PIPES buffer (P1851, Sigma), permeabilized with 0.1% Triton X-100 (T8787, Sigma) in 1×PBS, and blocked with 2% BSA. Primary and secondary antibodies were used as follows: anti-NSP2/3 (1:1500), anti-dsRNA (1:500), anti-ISG54 (1:500), anti-nucleocapsid (1:500), donkey anti-rabbit IgG alexafluor 488 (1:1000) (A-21441, Invitrogen), and goat anti-mouse IgG Alexa Fluor 568 (1:1000) (A11004, Thermofisher). Nuclei were visualized with Hoescht 33342 (1:2500) (H1339, Life Technologies). Cells were imaged by collecting Z-stack images with a Deltavision wide field fluorescent microscope (Applied Precision, GE) equipped with a digital camera (CoolSNAP HQ, Photometrics). Images were taken with a 20× or 100× lens. Samples were excited with light generated by an Insight SSI solid state illumination module (Applied Precision, GE) and deconvolved with SoftWoRx deconvolution software (Applied Precision, GE). All images were collected under identical acquisition conditions and processed using Imaris 7.6.4 (Bitplane).

FACS analysis. BMDMs (6.0×10⁵) were plated in a 12-well plate (Corning) for 24 h and subsequently infected with wild-type or mutant virus at an MOI of 0.1. At 6 hpi, cells were collected and fixed with 4% formaldehyde in 1×PBS, permeabilized with 0.1% Triton X-100 in 1×PBS, and blocked with 1×PBS containing 2% FCS and 0.5% sodium azide. Cells were labelled with fixable viability dye (65-0865-14, eBioscience) and antibodies against indicated primary and secondary antibodies, donkey anti-rabbit alexafluor 488 and goat anti-mouse primary antibody labeled with Alexa Fluor 568. Cells were analyzed using a LSR Fortessa cell analyzer (BD Bioscience). Flow cytometry data were analyzed using FlowJo software (Treestar).

Quantification of IFN-α production by reverse transcription quantitative PCR (RT-qPCR) and ELISA. BMDMs in a 12 or 24-well plate were mock-infected or infected with virus at an MOI of 0.1 or 1. At indicated time points, monolayer cells were used for RNA extraction using an RNeasy Mini Kit (74104, QIAGEN), while cell culture supernatants were collected for ELISA. To determine IFN-α11, beta-actin or MHV-A59 N gene mRNA production, total RNAs were extracted and an equal amount of RNA (˜1 μg) was used for cDNA synthesis using Rt² HT First Strand Kit (330401, QIAGEN). Quantitative PCR was performed with specific primers for mouse IFN-α11 (PPM03050B-200, QIAGEN), mouse β-actin (PPM02945B-200, QIAGEN) or MHV-A59 N gene using RT² SYBR Green qPCR Mastermix (330502, QIAGEN) in the Bio-Rad CFX96 system. Thermocycler was set as follows: one step at 95° C. (10 min), 40 cycles of 95° C. (15 s), 60° C. (1 min) and plate read, one step at 95° C. (10 s) and a melt curve from 65° C.-95° C. at increments of 0.5° C./0.05 s. Samples were evaluated in triplicate and data are representative of 3 independent experiments. To measure secreted IFN-α, 50 μL cell culture supernatant was used for assay using a mouse IFN-α ELISA kit (BMS6027, eBioscience) as per the manufacturer's instruction. Graphs were generated using Prism 7 software (GraphPad Software, Inc.).

Electron microscopy. 6.0×10⁵ B6 BMDMs were plated per well in a 12-well plate (Corning) in 1 mL BMM media. After 24 h, cells were infected with virus at 0.1 MOI in serum-free DMEM. Control cells were treated with BMM media. Following a 1 h incubation at 37° C./5% CO₂, media was replaced with BMM media. Uninfected controls were treated with 1 μM staurosporine (ALX-380-014, Enzo Life Sciences) prepared in BMM media to induce apoptosis. Cells were further incubated at 37° C./5% CO₂ for 16 h then prepped for EM. Cells were washed in 1×PBS then incubated for 30 min at 4° C. in 1×PBS. Cells were gently collected from plates using a pipette, pelleted at 1200 rpm for 5 min at 4° C. and fixed (4% glutaraldehyde in 0.1 M cacodylate buffer). Cell sections were prepared and imaged by the Electron Microscopy Core Facility at Loyola University Chicago, Maywood, IL.

Western blotting. 12-well plates (Corning) containing 6.0×10⁵ B6 BMDMs/well in BMM media were media-treated or infected with virus at 0.1 MOI in serum-free DMEM for 1 hour at 37° C./5% CO₂. Media was replaced with fresh BMM media and cells were returned to the incubator. After 6, 12 or 24 hours, cells were lysed in 100 μL cold lysis buffer (20 mM tris pH 7.5, 150 mM NaCl, 1 mM EGTA, 1 mM EDTA, 1% Triton X-100, 2.5 mM sodium pyrophosphate, 1 mM β-glycerophosphate, 1 mM sodium ortho-vanadate, 1 μg/mL leupeptin, 1 mM PSMF) and scraped into tubes. Cells in lysis buffer were incubated on ice for 10 min then pelleted at 14,000 rpm for 10 min at 4° C. Cell-free supernatant was diluted in 2× sample buffer (10% glycerol, 5% B-ME, 3% SDS, 7.5 mg/mL Trizma-base, bromophenol blue). Samples were separated by electrophoresis through 12% acrylamide PAGE-gel and transferred to PVDF Immuno-Blot membranes (162-0177, Bio-Rad) using the semi-dry transfer system (Bio-Rad). Membranes were suspended in blocking buffer, containing 5% w/v nonfat dry milk in 1×TBST (Tris-buffered saline+1% Tween-20), for 1.5 h at RT. To detect activated caspase-3, rabbit monoclonal antibody to cleaved caspase-3 (Asp175; Cell Signaling Technology) was applied (1:1000) overnight at 4° C. with gentle shaking. A second set of membranes, used to determine cell protein expression via mouse anti-βactin (1:5000) or relative virus replication via mouse-anti-J3.3 N protein (1:200), were incubated for 1.5 h at RT. All membranes were washed three times in 1×TBST and secondary antibody, donkey anti-rabbit-HRP (1:2500) for caspase-3 monoclonal or goat anti-mouse-HRP (1:5000) for N protein and B-actin, was applied for 1.5 h at RT with gentle shaking. Membranes were washed three times with 1×TBST and chemiluminescence was visualized using Western Lightning Plus-ECL reagent (50-904-9326, PerkinElmer, Inc.).

Purification of the recombinant MHV Nsp15 protein from E. coli. The cDNA sequences of WT MHV Nsp15 and T98M were amplified using the appropriate primers and subcloned into the pET-His-SUMO vector using the fusion PCR. The constructs harboring the WT Nsp15 or T98M mutant gene were transformed into Rosetta (DE3) pLys cells. A single colony was inoculated in 100 mL of Terrific Broth (TB) in the presence of 50 μg/mL of ampicillin and 17 μg/mL chloramphenicol and incubated at 37° C. overnight. The culture was then transferred into 3 L of TB media and cultured until the OD₆₀₀ reached 0.8. The temperature was decreased to 16° C. and IPTG was added to a final concentration of 0.2 mM for 20 h. The cells were collected by centrifugation at 8000×g for 10 min and cell pellets were suspended in lysis buffer containing 10% glycerol, 50 mM HEPES (pH 7.0), 400 mM NaCl, 5 mM β-ME and 10 mM imidazole. The lysate was centrifuged at 15,000×g for 30 min and the supernatant was loaded onto a Ni-NTA column. The Ni-NTA column was subsequently washed three times with lysis buffer containing 20 mM imidazole. The protein was eluted with elution buffer containing 500 mM imidazole and then the buffer was changed to 10% glycerol, 20 mM Tris-Cl (pH 7.5) and 5 mM β-ME. The sample was further purified by Mono Q-Sepharose with a gradient of Tris buffer containing 0 to 1 M NaCl. Nsp15 was quantified by SDS-PAGE compared to a known concentration of BSA, and stored at −80° C. in a buffer containing 10% glycerol, 20 mM Tris-CI, pH 7.5, 300 mM NaCl and 10 mM β-ME.

Dynamic light scattering (DLS). Recombinant MHV Nsp15 or T98M proteins were diluted in storage buffer (10% glycerol, 20 mM Tris-Cl, pH 7.5, 300 mM NaCl and 5 mM β-ME) at different concentrations. Size measurement was carried out by Zetasizer Nano-S dynamic light scattering (Malvern Instruments) at RT. Each sample was measured at least three times. The average intensity and size distributions are shown.

Differential scanning fluorimetry (DSF) assay. DSF was carried out in the Stratagene MX3005P real-time PCR machine. The samples contain 1× SYPRO Orange, 10 μM of the recombinant protein. All samples were heated at a rate of 0.5° C./min, and the fluorescence intensity and Tm (melting temperature) were determined.

Bioanalyzer RNA analysis. Equal amounts of total RNA purified from BMDMs were analyzed on an Agilent 2100 Bioanalyzer using RNA Nano LabChips.

Mouse experiments. All experiments were performed using protocols reviewed and approved by the Loyola University Chicago IACUC. C57BL/6J mice were purchased from Jackson Laboratory. For intracranial (i.e.) infections, six-week-old mice were inoculated with 600 PFU in 20 μL of WT or mutant MHV. Infected mice were monitored for body weight daily and euthanized when weight loss was over 25% according to the IACUC protocol. Graphs of survival rate were generated using Prism 7 software (GraphPad Software, Inc.). Statistical analysis of survival rate was conducted with log rank test. For intraperitoneal (i.p.) infection, six-week-old mice were injected with 60,000 PFU in 100 μL 1×PBS. Organs were collected at indicated time points and evaluated for viral replication. Evidence of viral pathogenesis was determined by H&E staining.

Sequence alignment. Nsp15 T98 region from representative strains of coronavirus sub-groups using Clustal W. Alpha-coronavirus: NL63 (Amsterdam I strain, AY567487), PEDV (CV777 strain, NC_003436); beta-coronavirus: MHV, (A59 strain, AY910861); SARS-COV (MA15 strain, FJ882957); MERS-COV (EMC strain, JX869059); gamma-coronavirus: IBV (Beaudette strain, NC_001451); delta-coronavirus: PDCoV (KJ567050).

While the invention has been described in terms of specific or particular embodiments and investigations, it should be apparent that alternatives could be adopted by one skilled in the art. For example, the specific mutated coronavirus could differ from that described or could have additional mutations. Accordingly, it should be understood that the invention is not necessarily limited to any embodiment described herein or illustrated in the drawings. It should also be understood that the phraseology and terminology employed above are for the purpose of describing the disclosed embodiments and investigations, and do not necessarily serve as limitations to the scope of the invention. Therefore, the scope of the invention is to be limited only by the following claims.

1. A live, attenuated coronavirus comprising a variant replicase gene encoding polyproteins comprising a non-structural protein (nsp)-15, the replicase gene encoding the nsp15 and causing any change, including mutations and/or deletions, that affects the stability or activity of the nsp15.

2. The coronavirus of claim 1, wherein the change comprises an amino acid mutation of threonine to methionine at position 98 or catalytic histidine to alanine at position 262.

3. The coronavirus of claim 1, wherein the coronavirus is a mutation of a wild-type coronavirus.

4. The coronavirus of claim 3, wherein the wild-type coronavirus is a Coronavirinae virus.

5. The coronavirus of claim 4, wherein the Coronavirinae virus is chosen from the group consisting of Severe Acute Respiratory Syndrome coronavirus (SARS-COV), Middle East respiratory syndrome coronavirus (MERS-COV), human coronaviruses 229E (HCoV-229E), OC43 (HCoV-OC43), HKU1 (HCoV-HKU1), and NL63 (HCoV-NL63), feline infectious peritonitis virus (FIPV), canine coronavirus (CCoV), infectious bronchitis virus (IBV) of chickens, bovine coronavirus (BoCoV), and porcine coronaviruses including transmissible gastroenteritis virus (TGEV), porcine delta coronavirus (PDCOV), porcine epidemic diarrhea virus (PEDV), porcine respiratory coronavirus (PRCV), and porcine hemagglutinating encephalomyelitis coronavirus (PHE-COV).

6. The variant replicase gene of claim 1.

7. A protein encoded by the variant replicase gene of claim 6.

8. A plasmid comprising the variant replicase gene of claim 6.

9. A vaccine comprising the coronavirus of claim 1 and a carrier.

10. A method for preventing a disease in a subject, the method comprising administering the vaccine of claim 9 to the subject.

11. The method of claim 10, wherein administering the vaccine causes activation of type I interferon in the subject which reduces the pathogenicity of the coronavirus relative to a coronavirus expressing a corresponding wild-type replicase gene.

12. A method of preventing a disease in a subject, the method comprising activating type I interferon in the subject, wherein activation of the type I interferon reduces the pathogenicity of a coronavirus.

13. A method of producing a vaccine, the method comprising:
modifying a wild-type coronavirus to produce a live, attenuated coronavirus comprising a variant replicase gene encoding polyproteins and causing a change, including mutations and/or deletions, in a non-structural protein (nsp)-15 that affects the stability or activity of the nsp15; and
producing a vaccine comprising the coronavirus and a carrier;
wherein administering the vaccine to a subject causes activation of type I interferon in the subject which reduces the pathogenicity of the wild-type coronavirus.

14. The method of claim 13, wherein the change comprises an amino acid mutation of threonine to methionine at position 98 or catalytic histidine to alanine at position 262.

15. The method of claim 13, wherein the wild-type coronavirus is a Coronavirinae virus.

16. The method of claim 13, wherein the Coronavirinae virus consists of Severe Acute Respiratory Syndrome coronavirus (SARS-COV), Middle East respiratory syndrome coronavirus (MERS-COV), human coronaviruses 229E (HCoV-229E), OC43 (HCoV-OC43), HKU1 (HCoV-HKU1), and NL63 (HCoV-NL63), feline infectious peritonitis virus (FIPV), canine coronavirus (CCoV), infectious bronchitis virus (IBV) of chickens, bovine coronavirus (BoCoV), and porcine coronaviruses including transmissible gastroenteritis virus (TGEV), porcine delta coronavirus (PDCOV), porcine epidemic diarrhea virus (PEDV), porcine respiratory coronavirus (PRCV), and porcine hemagglutinating encephalomyelitis coronavirus (PHE-COV).

17. The method of claim 16, further comprising administering the vaccine to the subject.

18. The method of claim 13, further comprising administering the vaccine to the subject.


SEQUENCE LISTING (15 sequences)

SEQ ID NO: 1 - protein, 5 aa, Mouse hepatitis virus
EQRKG

SEQ ID NO: 2 - protein, 5 aa, Mouse hepatitis virus
SSTYK

SEQ ID NO: 3 - protein, 5 aa, Synthetic construct
EQAKG

SEQ ID NO: 4 - protein, 5 aa, Synthetic construct
SSMYK

SEQ ID NO: 5 - protein, 5 aa, Synthetic construct
EQAKG

SEQ ID NO: 6 - protein, 5 aa, Synthetic construct
SSTYK

SEQ ID NO: 7 - protein, 12 aa, Mouse hepatitis virus
FCSSTYKVCKYT

SEQ ID NO: 8 - protein, 12 aa, SARS coronavirus
AHVSTIGVCTMT

SEQ ID NO: 9 - protein, 12 aa, MERS coronavirus
YGTATIGVCKYT

SEQ ID NO: 10 - protein, 12 aa, Human coronavirus NL63
FTCYTHSVCKYT

SEQ ID NO: 11 - protein, 12 aa, Porcine epidemic diarrhea virus
LTTFTKDVCKYT

SEQ ID NO: 12 - protein, 12 aa, Infectious bronchitis virus
LYRNTVKVCAYT

SEQ ID NO: 13 - protein, 12 aa, Porcine delta coronavirus
IFQYTINVSTYT

SEQ ID NO: 14 - RNA, 16 nt, Synthetic construct
  source 1..16: mol_type = other RNA
gaagcgaaaccctaag

SEQ ID NO: 15 - DNA, 31365 bp, Synthetic construct
  source 1..31365: mol_type = other DNA
gataagagtgattggcgtccgtacgtaccctctcaactctaaaactcttgtagtttaaatctaatctaaactttataaacggcacttcctgcgtgtccatgcccgcgggcctggtcttgtcatagtgctgacatttgtagttccttgactttcgttctctgccagtgacgtgtccattcggcgccagcagcccacccataggttgcataatggcaaagatgggcaaatacggtctcggcttcaaatgggccccagaatttccatggatgcttccgaacgcatcggagaagttgggtaaccctgagaggtcagaggaggatgggttttgcccctctgctgcgcaagaaccgaaagttaaaggaaaaactttggttaatcacgtgagggtgaattgtagccggcttccagctttggaatgctgtgttcagtctgccataatccgtgatatttttgtagatgaggatccccagaaggtggaggcctcaactatgatggcattgcagttcggtagtgccgtcttggttaagccatccaagcgcttgtctattcaggcatggactaatttgggtgtgcttcccaaaacagctgccatggggttgttcaagcgcgtctgcctgtgtaacaccagggagtgctcttgtgacgcccacgtggcctttcacctttttacggtccaacccgatggtgtatgcctgggtaatggccgttttataggctggttcgttccagtcacagccataccggagtatgcgaagcagtggttgcaaccctggtccatccttcttcgtaagggtggtaacaaagggtctgtgacatccggccacttccgccgcgctgttaccatgcctgtgtatgactttaatgtagaggatgcttgtgaggaggttcatcttaacccgaagggtaagtactcctgcaaggcgtatgctcttcttaagggctatcgcggtgttaagcccatcctgtttgtggaccagtatggttgcgactatactggatgtctcgccaagggtcttgaggactatggcgatctcaccttgagtgagatgaaggagttgttccctgtgtggcgtgactccttggatagtgaagtccttgtggcttggcacgttgatcgagatcctcgggctgctatgcgtctgcagactcttgctactgtacgttgcattgattatgtgggccaaccgaccgaggatgtggtggatggagatgtggtagtgcgtgagcctgctcatcttctcgcagccaatgccattgttaaaagactcccccgtttggtggagactatgctgtatacggattcgtccgttacagaattctgttataaaaccaagctgtgtgaatgcggttttatcacgcagtttggctatgtggattgttgtggtgacacctgcgattttcgtgggtgggttgccggcaatatgatggatggctttccatgtccagggtgtaccaaaaattatatgccctgggaattggaggcccagtcatcaggtgttataccagaaggaggtgttctattcactcagagcactgatacagtgaatcgtgagtcctttaagctctacggtcatgctgttgtgccttttggttctgctgtgtattggagcccttgcccaggtatgtggcttccagtaatttggtcttctgttaagtcatactctggtttgacttatacaggagtagttggttgtaaggcaattgttcaagagacagacgctatatgtcgttctctgtatatggattatgtccagcacaagtgtggcaatctcgagcagagagctatccttggattggacgatgtctatcatagacagttgcttgtgaataggggtgactatagtctcctccttgagaatgtggatttgtttgttaagcggcgcgctgaatttgcttgcaaattcgccacctgtggagatggtcttgtacccctcctactagatggtttagtgccccgcagttattatttgattaagagtggtcaagctttcacctctatgatggttaattttagccatgaggtgactgacatgtgtatggacatggctttattgttcatgcatgatgttaaagtggccactaagtatgttaagaaggttactggcaaactggccgtgcgctttaaagcgttgggtgtagccgttgtcagaaaaattactgaatggtttgatttagccgtggacattgctgctagtgccgctggatggctttgctaccagctggtaaatggcttatttgcagtggccaatggtgttataacctttgtacaggaggtgcctgagcttgtcaagaattttgttgacaagttcaaggcatttttcaaggttttgatcgactctatgtcggtttctatcttgtctggacttactgttgtcaagactgcctcaaatagggtgtgtcttgctggcagtaaggtttatgaagttgtgcagaaatctttgtctgcatatgttatgcctgtgggttgcagcgaagccacttgtttggtgggtgagattgaacctgcagtttttgaagatgatgttgttgatgtggttaaagccccattaacatatcaaggctgttgtaagccacccacttctttcgagaagatttgtattgtggataaattgtatatggccaagtgtggtgatcaattttaccctgtggttgttgataacgacactgttggcgtgttagatcagtgctggaggtttccctgtgcgggcaagaaagtcgagtttaacgacaagcccaaagtcaggaagataccctccacccgtaagattaagatcaccttcgcactggatgcgacctttgatagtgttctttcgaaggcgtgttcagagtttgaagttgataaagatgttacattggatgagctgcttgatgttgtgcttgacgcagttgagagtacgctcagcccttgtaaggagcatgatgtgataggcacaaaagtttgtgctttacttgataggttggcaggagattatgtctatctttttgatgagggaggcgatgaagtgatcgccccgaggatgtattgttccttttctgctcctgatgatgaagactgcgttgcagcggatgttgtagatgcagatgaaaaccaagatgatgatgctgaagactcagcagtccttgtcgctgatacccaagaagaggacggcgttgccaaggggcaggttgaggcggattcggaaatttgcgttgcgcatactggtagtcaagaagaattggctgagcctgatgctgtcggatctcaaactcccatcgcctctgctgaggaaaccgaagtcggagaggcaagcgacagggaagggattgctgaggcgaaggcaactgtgtgtgctgatgctgtagatgcctgccccgatcaagtggaggcatttgaaattgaaaaggttgaagactctatcttggatgagcttcaaactgaacttaatgcgccagcggacaagacctatgaggatgtcttggcattcgatgccgtatgctcagaggcgttgtctgcattctatgctgtgccgagtgatgaaacccactttaaagtgtgtggattctattcgcctgctatagagcgcactaattgttggctgcgttctactttgatagtaatgcagagtctacctttggaatttaaagacttggagatgcaaaagctctggttgtcttacaaggccggctatgaccaatgctttgtggacaaactagttaagagcgtgcccaagtctattatccttccacaaggtggttatgtggcagattttgcctatttctttctaagccagtgtagctttaaagcttatgctaactggcgttgtttagagtgtgacatggagttaaagcttcaaggcttggacgccatgtttttctatggggacgttgtgtctcatatgtgcaagtgtggtaatagcatgaccttgttgtctgcagatataccctacactttgcattttggagtgcgagatgataagttttgcgctttttacacgccaagaaaggtctttagggctgcttgtgcggtagatgttaatgattgtcactctatggctgtagtagagggcaagcaaattgatggtaaagtggttaccaaatttattggtgacaaatttgattttatggtgggttacgggatgacatttagtatgtctccttttgaactcgcccagttatatggttcatgtataacaccaaatgtttgttttgttaaaggagatgttataaaggttgttcgcttagttaatgctgaagtcattgttaaccctgctaatgggcgtatggctcatggtgcaggtgttgcaggtgctatagctgaaaaggcgggcagtgcttttattaaagaaacctccgatatggtgaaggctcagggcgtttgccaggttggtgaatgctatgaatctgccggtggtaagttatgtaaaaaggtgcttaacattgtagggccagatgcgcgagggcatggcaagcaatgctattcacttttagagcgtgcttatcagcatattaataagtgtgacaatgttgtcactactttaatttcggctggtatatttagtgtgcctactgatgtctccctaacttacttacttggtgtagtgacaaagaatgtcattcttgtcagtaacaaccaggatgattttgatgtgatagagaagtgtcaggtgacctccgttgctggtaccaaagcgctatcacttcaattggccaaaaatttgtgccgtgatgtaaagtttgtgacgaatgcatgtagttcgctttttagtgaatcttgctttgtctcaagctatgatgtgttgcaggaagttgaagcgctgcgacatgatatacaattggatgatgatgctcgtgtctttgtgcaggctaatatggactgtctgcccacagactggcgactggttaacaaatttgatagtgttgatggtgttagaaccattaagtattttgaatgcccgggcgggatttttgtatccagccagggcaaaaagttcggttatgttcagaatggttcatttaaggaggcgagtgttagccaaataagggctttactcgctaataaggttgatgtcttgtgtactgttgatggtgttaacttccgctcctgctgcgtagcagagggtgaagtttttggcaagacattaggttcagtcttttgtgatggcataaatgtcaccaaagttaggtgtagtgccatttacaagggtaaggttttctttcagtacagtgatttgtccgaggcagatcttgtggctgttaaagatgcctttggttttgatgaaccacaactgctgaagtactacactatgcttggcatgtgtaagtggccagtagttgtttgtggcaattattttgctttcaagcagtcaaataataattgctatataaatgtggcatgtttaatgctgcaacacttgagtttaaagtttcctaagtggcaatggcaagaggcttggaacgagttccgctctggtaaaccactaaggtttgtgtccttggtattagcaaagggcagctttaaatttaatgaaccttctgattctatcgattttatgcgtgtggtgctacgtgaagcagatttgagtggtgccacgtgcaatttggaatttgtttgtaaatgtggtgtgaagcaagagcagcgcaaaggtgttgacgctgttatgcattttggtacgttggataaaggtgatcttgtcaggggttataatatcgcatgtacgtgcggtagtaaacttgtgcattgcacccaatttaacgtaccatttttaatttgctccaacacaccagagggtaggaaactgcccgacgatgttgttgcagctaatatttttactggtggtagtgtgggccattacacgcatgtgaaatgtaaacccaagtaccagctttatgatgcttgtaatgttaataaggtttcggaggctaagggtaattttaccgattgcctctaccttaaaaatttaaagcaaactttttcgtctgtgctgacgactttttatttagatgatgtaaagtgtgtggagtataagccagatttatcgcagtattactgtgagtctggtaaatattatacaaaacccattattaaggcccaatttagaacatttgagaaggttgatggtgtctataccaactttaaattggtgggacatagtattgctgaaaaactcaatgctaagctgggatttgattgtaattctccctttgtggagtataaaattacagagtggccaacagctactggagatgtggtgttggctagtgatgatttgtatgtaagtcggtactcaagcgggtgcattacttttggtaaaccggttgtctggcttggccatgaggaagcatcgctgaaatctctcacatattttaatagacctagtgtcgtttgtgaaaataaatttaatgtgttgcccgttgatgtcagtgaacccacggacaaggggcctgtgcctgctgcagtccttgttaccggcgtccctggagctgatgcgtcagctggtgccggtattgccaaggagcaaaaagcctgtgcttctgctagtgtggaggatcaggttgttacggaggttcgtcaagagccatctgtttcagctgctgatgtcaaagaggttaaattgaatggtgttaaaaagcctgttaaggtggaaggtagtgtggttgttaatgatcccactagcgaaaccaaagttgttaaaagtttgtctattgttgatgtctatgatatgttcctgacagggtgtaagtatgtggtttggactgctaatgagttgtctcgactagtaaattcaccgactgttagggagtatgtgaagtggggtatgggaaagattgtaacacccgctaagttgttgttgttaagagatgagaagcaagagttcgtagcgccaaaagtagtcaaggcgaaagctattgcctgctattgtgctgtgaagtggtttctcctctattgttttagttggataaagtttaatactgataataaggttatatacaccacagaagtagcttcaaagcttactttcaagttgtgctgtttggcctttaagaatgccttacagacgtttaattggagcgttgtgtctaggggctttttcctagttgcaacggtctttttattatggtttaactttttgtatgctaatgttattttgagtgacttctatttgcctaatattgggcctctccctacgtttgtgggacagatagttgcgtggtttaagactacatttggtgtgtcaaccatctgtgatttctaccaggtgacggatttgggctatagaagttcgttttgtaatggaagtatggtatgtgaactatgcttctcaggttttgatatgctggacaactatgatgctataaatgttgttcaacacgttgtagataggcgtttgtcctttgactatattagcctatttaaattagtagttgagcttgtaatcggctactctctttatactgtgtgcttctacccactgtttgtccttattggaatgcagttgttgaccacatggttgcctgaattctttatgctggagactatgcattggagtgctcgtttgtttgtgtttgttgccaatatgcttccagcttttacgttactgcgattttacatcgtggtgacagctatgtataaggtctattgtctttgtagacatgttatgtatggatgtagtaagcctggttgcttgttttgttataagagaaaccgtagtgtccgtgttaagtgtagcaccgttgttggtggttcactacgctattacgatgtaatggctaacggcggcacaggtttctgtacaaagcaccagtggaactgtcttaattgcaattcctggaaaccaggcaatacattcataactcatgaagcagcggcggacctctctaaggagttgaaacgccctgtgaatccaacagattctgcttattactcggtcacagaggttaagcaggttggttgttccatgcgtttgttctacgagagagatggacagcgtgtttatgatgatgtcaatgctagtttgtttgtggacatgaatggtctgctgcattctaaagttaaaggtgtgcctgaaacgcatgttgtggttgttgagaatgaagctgataaagctggttttctcggcgccgcagtgttttatgcacaatcgctctacagacctatgttgatggtggaaaagaaattaataactaccgccaacactggtttgtctgttagtcgaactatgtttgacctttatgtagattcattgctgaacgtcctcgacgtggatcgcaagagtctaacaagttttgtaaatgctgcgcacaactctctaaaggagggtgttcagcttgaacaagttatggatacctttattggctgtgcccgacgtaagtgtgctatagattctgatgttgaaaccaagtctattaccaagtccgtcatgtcggcagtaaatgctggcgttgattttacggatgagagttgtaataacttggtgcctacctatgttaaaagtgacactatcgttgcagccgatttgggtgttcttattcagaataatgctaagcatgtacaggctaatgttgctaaagccgctaatgtggcttgcatttggtctgtggatgcttttaaccagctatctgctgacttacagcataggctgcgaaaagcatgttcaaaaactggcttgaagattaagcttacttataataagcaggaggcaaatgttcctattttaactacaccgttctctcttaaagggggcgctgtttttagtagaatgttacaatggttgtttgttgctaatttgatttgtttcattgtgttgtgggcccttatgccaacatatgcagtgcacaaatcggatatgcagttgcctttatatgccagttttaaagttatagataatggtgtgctaagggatgtgtctgttactgacgcatgcttcgcaaacaaatttaatcaatttgatcaatggtatgagtctacttttggtcttgcttattaccgcaactctaaggcttgtcctgttgtggttgctgtaatagatcaagacattggccataccttatttaatgttcctaccacagttttaagatatggatttcatgtgttgcattttataacccatgcatttgctactgatagcgtgcagtgttacacgccacatatgcaaatcccctatgataatttctatgctagtggttgcgtgttgtcatccctctgtactatgcttgcgcatgcagatggaaccccgcatccttattgttatacagggggtgttatgcacaatgcctctctgtatagttctttggctcctcatgtccgttataacctggctagttcaaacggttatatacgttttcccgaagtggttagtgaaggcattgtgcgtgttgtgcgcactcgctctatgacctactgcagggttggtttatgtgaggaggccgaggagggtatctgctttaattttaatcgttcatgggtattgaacaacccgtattatagggccatgcctggaactttttgtggtaggaatgcttttgatttaatacatcaagttttaggaggattagtgcggcctattgatttctttgccttaacggcgagttcagtggctggtgctatccttgcaattattgtcgttttggctttctattatttaataaagcttaaacgtgcctttggtgactacactagtgttgtggttatcaatgtaattgtgtggtgtataaattttctgatgctttttgtgtttcaggtttatcccacattgtcttgtttatatgcttgtttttatttctacacaacgctttatttcccttcggagataagtgttgttatgcatttgcaatggcttgtcatgtatggtgctattatgcccttgtggttttgcattatttacgtggcagtcgttgtttcaaaccatgcattgtggttgttctcttactgccgcaaaattggtaccgaggttcgtagtgacggcacatttgaggaaatggcccttactacctttatgattactaaagaatcttattgtaagttgaaaaattctgtttctgatgttgcttttaacaggtacttgagtctttataacaagtatcgttattttagtggcaaaatggatactgccgcttatagagaggctgcctgttcacaactggcaaaggcaatggaaacatttaaccataataatggtaatgatgttctctatcagcctccaaccgcctctgttactacatcatttttacagtctggtatagtgaagatggtgtcgcccacctctaaagtggagccttgtattgttagtgttacttatggtaacatgacacttaatgggttgtggttggatgataaagtttattgcccaagacatgttatctgttcttcagctgacatgacagaccctgattatcctaatttgctttgtagagtgacatcaagtgatttttgtgttatgtctggtcgtatgagccttactgtaatgtcttatcaaatgcagggctgccaacttgttttgactgttacactgcaaaatcctaacacgcctaagtattccttcggtgttgttaagcctggtgagacatttactgtactggctgcatacaatggcagacctcaaggagccttccatgttacgcttcgtagtagccataccataaagggctcctttctatgtggatcctgcggttctgtaggatatgttttaactggcgatagtgtacgatttgtttatatgcatcagctagagttgagtactggttgtcataccggtactgactttagtgggaacttttatggtccctatagagatgcgcaagttgtacaattgcctgttcaggattatacgcagactgttaatgttgtagcttggctttatgctgctatttttaacagatgcaactggtttgtgcaaagtgatagttgttccctggaggagtttaatgtttgggctatgaccaatggttttagctcaatcaaagccgatcttgtcttggatgcgcttgcttctatgacaggcgttacagttgaacaggtgttggccgctattaagaggctgcattctggattccagggcaaacaaattttaggtagttgtgtgcttgaagatgagctgacaccaagtgatgtttatcaacaactagctggtgtcaagctacagtcaaagcgcacaagagttataaaaggtacatgttgctggatattggcttcaacgtttttgttctgtagcattatctcagcatttgtaaaatggactatgtttatgtatgttactacccatatgttgggagtgacattgtgtgcactttgttttgtaagctttgctatgttgttgatcaagcataagcatttgtatttaactatgtatattatgcctgtgttatgcacactgttttacaccaactatttggttgtgtacaaacagagttttagaggtctagcttatgcttggctttcacactttgtccctgctgtagattatacatatatggatgaagttttatatggtgttgtgttgctagtagctatggtgtttgttaccatgcgtagcataaaccacgacgtcttttctattatgttcttggttggtagacttgtcagcctggtatccatgtggtattttggagccaatttagaggaagaggtactattgttcctcacatccctatttggcacgtacacatggactactatgttgtcattggctaccgctaaggttattgctaaatggttggctgtgaatgtcttgtacttcacagacgtaccgcaaattaaattagttcttttgagctacttgtgtattggttatgtgtgttgttgttattggggaatcttgtcactccttaatagcatttttaggatgccattgggcgtctacaattataaaatctccgttcaggagttacgttatatgaatgctaatggcttgcgcccacctagaaatagttttgaggccctgatgcttaattttaagctgttgggaattggtggtgtgccagtcattgaagtatctcaaattcaatcaagattgacggatgttaaatgtgctaatgttgtgttgcttaattgcctccagcacttgcatattgcatctaattctaagttgtggcagtattgtagtactttgcacaatgaaatactggctacatctgatttgagcgtggccttcgataagttggctcagctcttagttgttttatttgctaatccagcagcagtggatagcaagtgccttgcaagtattgaagaagtgagcgatgattacgttcgcgacaatactgtcttgcaagccttacagagtgaatttgttaatatggctagcttcgttgagtatgaacttgctaagaagaatctagatgaggctaaggctagcggctctgccaatcaacagcagattaagcagctagagaaggcgtgtaatattgctaagtcagcatatgagcgcgacagagctgttgctcgtaagctggaacgtatggctgatttagctcttacaaacatgtataaagaagctagaattaatgataagaagagtaaggtagtgtctgcattgcaaaccatgctctttagtatggtgcgtaagctagataaccaagctcttaattctattttagataatgcagttaagggttgtgtacctttgaatgcaataccatcattgacttcgaacactctgactataatagtgccagataagcaggtttttgatcaggttgtggataatgtgtatgtcacctatgctgggaatgtatggcatatacagtttattcaagatgctgatggtgctgttaaacaattgaatgagatagatgttaattcaacctggcctctagtcattgctgcaaataggcataatgaagtgtctactgttgttttgcagaacaatgagttgatgcctcagaagttgagaactcaggttgtcaatagtggctcagatatgaattgtaatactcctacccagtgttactataatactactggcacgggtaagattgtgtatgctatcctaagtgactgtgatggtctcaagtacactaagatagtaaaagaagatggaaattgtgttgttttggaattggatcctccctgtaagttttctgttcaggatgtgaagggccttaaaattaagtacctttactttgtgaaggggtgtaatacactggctagaggctgggttgtaggcaccttatcctcgacagtgagattgcaggcgggtacggcaactgagtatgcctccaactctgcaatactgtcgctgtgtgcgttttctgtagatcctaagaaaacgtacttggattatataaaacagggtggagttcccgttactaattgtgttaagatgttatgtgaccatgctggcactggtatggccattactattaagccggaggcaaccactaatcaggattcttatggtggtgcttccgtttgtatatattgccgctcgcgtgttgaacatccagatgttgatggattgtgcaaattacgcggcaagtttgtccaagtgcccttaggcataaaagatcctgtgtcatatgtgttgacgcatgatgtttgtcaggtttgtggcttttggcgagatggtagctgttcctgtgtaggcacaggctcccagtttcagtcaaaagacacgaactttttaaacgggttcggggtacaagtgtaaatgcccgtcttgtaccctgtgccagtggcttggacactgatgttcaattaagggcatttgacatttgtaatgctaatcgagctggcattggtttgtattataaagtgaattgctgccgcttccagcgtgtagatgaggacggcaacaagttggataagttctttgttgttaaaagaactaatttagaagtgtataataaggagaaagaatgctatgagttgacaaaagaatgcggtgttgtggctgaacacgagttcttcacatttgatgtggagggaagtcgggtaccacacatagtccgtaaagatctttcaaagtttactatgttagatctttgctatgcattgcgtcattttgaccgcaatgattgttcaactcttaaggaaattctacttacatatgctgagtgtgaagagtcctacttccaaaagaaggactggtatgattttgttgagaatcctgatataattaatgtgtataaaaagcttggtcctatatttaatagagccctgcttaacactgccaagtttgcagacgcattagtggaggcaggcttagtaggtgttttaacacttgataatcaagatttatatggtcaatggtatgactttggagattttgtcaagacagtacctggttgtggtgttgccgtggcagactcttattattcatatatgatgccaatgctgactatgtgtcatgcgttggatagtgagttgtttgttaatggtacttatagggagtttgaccttgttcagtatgattttactgatttcaagctagagctcttcactaagtattttaagcattggagtatgacctaccacccgaacacctgtgagtgcgaggatgacaggtgcattattcattgcgccaattttaatatactttttagtatggtcttacctaagacctgttttgggcctcttgttaggcagatatttgtggatggtgttcctttcgttgtgtcgatcggttaccattataaagaattaggtgttgttatgaatatggatgtggatacacatcgttatcgcttgtctcttaaggacttgcttttgtatgctgcagaccctgcccttcatgtggcgtctgctagtgcactgcttgatttgcgcacatgttgttttagcgttgcagctattacaagtggcgtaaaatttcaaacagttaaacctggaaattttaatcaggatttttatgagtttattttgagtaaaggcctgcttaaagaggggagctccgttgatttgaagcacttcttctttacgcaggatggtaatgctgctattactgattataattattacaagtataatctacccaccatggtggatattaagcagttgttgtttgttttagaagttgttaataagtattttgagatctatgagggtgggtgtatacccgcaacacaggtcattgttaataattatgataagagtgctggctatccatttaataaatttggaaaggccaggctctattatgaggcattatcatttgaggagcaggatgaaatttatgcgtataccaaacgcaatgtcctgccgaccctaactcaaatgaatcttaaatatgctattagtgctaagaatagggcccgcaccgttgctggtgtctctattctcagtactatgactggcagaatgtttcatcaaaagtgtctaaagagtatagcagctactcgcggtgttcctgtagttataggcaccacgaagttctatggcggttgggatgatatgttacgccgccttattaaagatgttgatagtcctgtactcatgggttgggactatcctaaatgtgatcgtgctatgccaaacatactgcgtattgttagtagtttggtgctagcccgtaaacatgattcgtgctgttcgcatacggatagattctatcgtcttgcgaacgagtgcgcccaagttttgagtgaaattgttatgtgtggtggttgttattatgttaaaccaggtggcactagtagtggggatgcaaccactgcttttgctaattctgtgtttaacatttgtcaagctgtttccgccaatgtatgctcgcttatggcatgcaatggacacaaaattgaagatttgagtatacgcgagttacaaaagcgcctatactctaatgtctatcgtgcggaccatgttgaccccgcatttgttagtgagtattatgagtttttaaataagcattttagtatgatgattttgagtgatgatggtgttgtgtgttataattcagagtttgcgtccaagggttatattgctaatataagtgcctttcaacaggtattatattatcaaaataatgtgtttatgtctgaggccaaatgttgggtagaaacagacatcgaaaagggaccgcatgaattttgttctcaacatacaatgctagtcaagatggatggtgatgaagtctaccttccataccctgatccttcgagaatcttaggagcaggctgttttgttgatgatttattaaagactgatagcgttctcttgatagagcgtttcgtaagtcttgcaattgatgcttatcctttagtataccatgagaacccagagtatcaaaatgtgttccgggtatatttagaatatataaagaagctgtacaatgatctcggtaatcagatcctggacagctacagtgttattttaagtacttgtgatggtcaaaagtttactgatgagaccttttacaagaacatgtatttaagaagtgcagtgctgcaaagcgttggtgcctgcgttgtctgtagttctcaaacatcattacgttgtggcagttgcatacgcaagcctttgctgtgttgcaaatgcgcctatgatcatgttatgtccactgatcataaatatgtcctgagtgtgtcaccatatgtgtgtaattcaccgggatgtgatgtaaatgatgttaccaaattgtatttaggtggtatgtcatattattgtgaggaccataaaccacagtattcattcaaattggtgatgaatggtatggtttttggtttatataaacaatcttgtactggttcgccctacatagaggattttaataaaatagctagttgcaaatggacagaagtcgatgattatgtgctagctaatgaatgcaccgaacgccttaaattgtttgccgcagaaacgcagaaggccacagaagaggcctttaagcaatgttatgcgtcagcaacgatccgtgagatcgtgagcgatcgggagttaattttatcttgggaaattggtaaagtgagaccaccacttaataaaaattatgtttttactggctaccattttactaataatggtaagacagttttaggtgagtatgtttttgataagagtgagttgactaatggtgtgtactatcgcgccacaaccacttataagttatctgtaggtgatgtgttcattttaacatcacacgcagtgtctagtttaagtgctcctacattagtaccgcaggagaattatactagcattcgttttgctagtgtttatagtgtgcctgaaacctttcagaataatgtgcctaattatcagcacattggaatgaagcgctattgtactgtacagggaccgcctggtactggtaagtcccatctagccattgggctagctgtttattattgtacagcgcgcgtggtgtataccgctgctagccatgctgcagttgacgcgctgtgtgaaaaggcacataaatttttaaatattaatgactgcacgcgtattgttcctgcaaaggtgcgtgtagattgttatgataaatttaaggtcaatgacaccactcgcaagtatgtgtttactacaataaatgcattacctgagttggtgactgacattattgtcgttgatgaggttagtatgcttaccaactatgagctgtctgttattaacagccgtgttagggctaagcattatgtgtatattggagaccctgcgcagttacctgcaccacgtgtgctactgaataagggaactctagaacctagatattttaattccgttaccaagctaatgtgttgtttgggtccagatattttcttgggcacctgttatagatgccctaaggagattgtggatacggtgtcagccttggtttataataataagctgaaggctaaaaatgataatagctccatgtgctttaaggtttattataagggccagactacacatgagagttctagtgctgttaatatgcagcaaatacatttaattagtaagtttttaaaggcaaaccccagttggagtaacgccgtatttattagtccttataatagtcagaactatgttgctaagagagtcttgggattacaaacccagacagtagactcagcgcagggttctgaatatgattttgttatttattcacagactgcggaaacagcgcattctgtcaatgtaaatagattcaatgttgctattacacgtgctaagaagggtattctctgtgtcatgagtagtatgcaattatttgagtctcttaattttactacactgacgttggataagattaacaatccacgattacagtgtactacaaatttgtttaaggattgtagcaggagctatgtaggatatcacccagcccatgcaccatcctttttggcagttgatgacaaatataaggtaggcggtgatttagccgtttgccttaatgttgctgattctgctgtcacttattcgcggcttatatcactcatgggattcaagcttgacttgacccttgatggttattgtaagctgtttataactagagatgaagctatcaaacgtgttagagcctgggttggcttcgatgcagaaggtgcccatgcgatacgtgatagcattgggacaaatttcccattacaattaggcttttcgactggaattgattttgttgtcgaagccactggaatgtttgctgagagagatggttatgtctttaaaaaggcagccgcacgagctcctcctggcgaacaatttaaacaccttatcccacttatgtcaagagggcagaaatgggatgtggttcgaattagaatagtacaaatgttgtcagaccacctagcggatttggcagacagtgttgtacttgtgacgtgggctgccagctttgagctcacatgtttgcgatatttcgctaaagttggaagagaagttgtgtgtagtgtctgcaccaagcgtgcgacatgttttaattctagaactggatactatggatgctggcgacatagttattcctgtgattacctgtacaacccactaatagttgacattcaacagtggggatatacaggatctttaactagcaatcatgatcctatttgcagcgtgcataagggtgctcatgttgcatcatctgatgctatcatgacccggtgtctagctgttcatgattgcttttgtaagtctgttaattggaatttagaataccccattatttcaaatgaggtcagtgttaatacctcctgcaggttattgcagcgcgtaatgtttagggctgcgatgctatgcaataggtatgatgtgtgttatgacattggcaaccctaaaggtcttgcctgtgtcaaaggatatgattttaagttttatgatgcctcccctgttgttaagtctgttaaacagtttgtttataaatacgaggcacataaagatcaatttttagatggtttgtgtatgttttggaactgcaatgtggataagtatccagcgaatgcagttgtgtgtaggtttgacacgcgtgtgttgaacaaattaaatctccctggctgtaatggtggcagtttgtatgttaacaaacatgcattccacaccagtccctttacccgggctgccttcgagaatttgaagcctatgcctttcttttattattcagatacgccctgtgtgtatatggaaggcatggaatctaagcaggtcgattatgtcccattgagaagcgctacatgcatcacaagatgcaatttaggtggcgctgtttgtttaaaacatgctgaggagtatcgtgagtaccttgagtcttacaatacggcaaccacagcgggttttactttttgggtctataagacttttgatttttataacctttggaatacttttactaggctccaaagtttagaaaatgtagtgtataatttggtcaatgctggacactttgatggccgggcgggtgaactgccttgtgctgttataggtgagaaagtcattgccaagattcaaaatgaggatgtcgtggtctttaaaaataacacgccattccccactaatgtggccgtcgaattatttgctaagcgcagtattcggccccaccccgagcttaagctctttagaaatttgaatattgacgtgtgctggagtcacgtcctttgggattatgctaaggatagtgtgttttgcagttcgacgtataaggtctgcaaatacacagatttacagtgcattgaaagcttgaatgtactttttgatggtcgtgataatggtgctcttgaagcttttaagaagtgccggaatggcgtctacattaacacgacaaaaattaaaagtctgtcgatgattaaaggcccacaacgtgccgatttgaatggcgtagttgtggagaaagttggagattctgatgtggaattttggtttgctgtgcgtaaagacggtgacgatgttatcttcagccgtacagggagccttgaaccgagccattaccggagcccacaaggtaatccgggtggtaatcgcgtgggtgatctcagcggtaatgaagctctagcgcgtggcactatctttactcaaagcagattattatcttctttcacacctcgatcagagatggagaaagattttatggatttagatgatgatgtgttcattgcaaaatatagtttacaggactacgcgtttgaagccgttgtttatggtagttttaaccagaagattattggaggtttgcatttgcttattggcttagcccgtaggcagcaaaaatccaatctggtaattcaagagttcgtgacatacgactctagcattcattcgtactttatcactgacgagaacagtggtagtagtaagagtgtgtgcactgttattgatttattgttagatgattttgtggacattgtaaagtccctgaatctaaagtgtgtgagtaaggttgttaatgttaatgttgattttaaagatttccagtttatgttgtggtgcaatgaggagaaggtcatgactttctatcctcgtttgcaggctgctgctgactggaaacctggttatgttatgcctgtcttatataagtatttggaatcgcctctggaaagagtaaacctctggaattatggcaagccgattactttacctacaggatgtatgatgaatgttgctaagtatactcaattatgtcaatatttgagcactacaacattagcagttccggctaatatgcgtgtcttacaccttggtgccggttcggataagggtgttgcccctgggtctgcagttcttaggcagtggctaccagcgggaagtattcttgtagataatgatgtgaatccatttgtgagtgacagtgtcgcctcatattatggaaattgtataaccttaccctttgattgtcagtgggatctgataatttctgatatgtacgaccctcttactaagaacattggggagtacaacgtgagtaaagatggattctttacttacctctgtcatttaattcgtgacaagttggctctgggtggcagtgttgccataaaaataacagagttttcttggaacgctgagttatatagtttaatggggaagtttgcgttctggacaatcttttgcaccaacgtaaacgcctcttcaagtgaaggatttttgattggcataaattggttgaataagacccgtaccgaaattgacggtaaaaccatgcatgccaattatctgttttggagaaatagtacaatgtggaatggaggggcttacagtctctttgacatgagtaagttccctttgaaagcggctggtacggctgttgttagccttaaaccagaccaaataaatgacttagtcctctccttgattgagaagggcaagttattagtgcgtgatacacgcaaagaagtttttgttggcgatagcctagtaaatgtcaaataaatctatacttgtcgtggctgtgaaaatggcctttgctgacaagcctaatcatttcataaactttcccctggcccaatttagtggctttatgggtaagtatttaaagctacagtctcaacttgtggaaatgggtttagactgtaaattacagaaggcaccacatgttagtattaccctgcttgatattaaagcagaccaatacaaacaggtggaatttgcaatacaagaaataatagatgatctggcggcatatgagggagatattgtctttgacaaccctcacatgcttggcagatgccttgttcttgatgttagaggatttgaagagttgcatgaagatattgttgaaattctccgcagaaggggttgcacggcagatcaatccagacactggattccgcactgcactgtggcccaatttgacgaagaaagagaaacaaaaggaatgcaattctatcataaagaacccttctacctcaagcataacaacctattaacggatgctgggcttgagctcgtgaagataggttcttccaaaatagatgggttttattgtagtgaactgagtgtttggtgtggtgagaggctttgttataagcctccaacacccaaattcagtgatatatttggctattgctgcatagataaaatacgtggtgatttagaaataggagacctaccgcaggatgatgaggaagcgtgggccgagctaagttaccactatcaaagaaacacctacttcttcagacatgtgcacgataatagcatctattttcgtaccgtgtgtagaatgaagggttgtatgtgttgatttgtttttacactattagtgtaataagcttattattttgttgaaaagggcgggatgtgcatagctatggctcctctcacactgcttttgctgatttgatgtcagctggtgtttgggttcaatgaacctcttaacatcgtttcacatttaaatgatgactggtttctatttggtgacagtcggtccgactgtacctatgtagaaaataacggtcatcctaaattagattggcttgacctcgacccaaagttgtgtaattcaggaaagatttccgcaaagagtggtaactctctctttaggagttttcacttcactgatttttacaattatacgggtgagggagaccaaattgtattttatgaaggagttaattttagtcccagccatggctttaaatgcctggctcatggagataataaaagatggatgggcaataaagctcgattttatgcccgagtgtatgagaagatggcccaatataggagcctatcgtttgttaatgtgtcttatgcctatggaggtaatgcaaagcccgcctccatttgcaaagacaatactttaacactcaataaccccaccttcatatcgaaggagtctaattatgttgattattactatgagagtgaggctaatttcacactagaaggttgtgatgaatttatagtaccgctctgtggttttaatggccattccaagggcagctcttcggatgctgccaataaatattatactgactctcagagttactataatatggatattggtgtcttatatgggttcaattcgaccttggatgttggcaacactgctaaggatccgggtcttgatctcacttgtaggtatcttgcattgactcctggtaattataaggctgtgtccttagagtatttgttaagcttaccctcaaaggctatttgcctccataagacaaagcgctttatgcctgtgcaggtagttgactcaaggtggagtagcatccgccagtcagacaatatgaccgctgcagcctgtcagctgccatattgtttctttcgcaacacatctgcgaattatagtggtggcacacatgatgcgcaccatggtgattttcatttcaggcagttattgtctggtttgttatataatgtttcctgtattgcccagcagggtgcatttctttataataatgttagttcctcttggccagcctatgggtacggtcattgtccaacggcagctaacattggttatatggcacctgtttgtatctatgaccctctcccggtcatactgctaggtgtgttattgggtatagctgtgttgattattgtgtttttgatgttttattttatgacggatagcggtgttagattgcatgaggcataatctaaacatgctgttcgtgtttattctatttttgccctcttgcctagggtatattggtgattttagatgtatccagcttgtgaattcaaacggtgctaatgttagtgctccaagcattagcactgagaccgttgaagtttcacaaggcctggggacatattatgtgttagatcgagtttatttaaatgccacattattgcttactggttactacccggtcgatggttctaagtttagaaacctcgctcttacgggaactaactcagttagcttgtcgtggtttcaaccaccctatttaagtcagtttaatgatggcatatttgcgaaggtgcagaaccttaagacaagtacgccatcaggtgcaactgcatattttcctactatagttataggtagtttgtttggctatacttcctataccgttgtaatagagccatataatggtgttataatggcctcagtgtgccagtataccatttgtcagttaccttacactgattgtaagcctaacactaatggtaataaattaatagggttttggcacacggatgtaaaacccccaatttgtgtgttaaagcgaaatttcacgcttaatgttaatgctgatgcattttattttcatttttaccaacatggtggtactttttatgcgtactatgcggataaaccctccgctactacgtttttgtttagtgtatatattggcgatattttaacacagtattatgtgttacctttcatctgcaacccaacagctggtagcacttttgctccgcgctattgggttacacctttggttaagcgccaatatttgtttaatttcaaccagaagggtgtcattactagtgctgttgattgtgctagtagttataccagtgaaataaaatgtaagacccagagcatgttacctagcactggtgtctatgagttatccggttatacggtccaaccagttggagttgtataccggcgtgttgctaacctcccagcttgtaatatagaggagtggcttactgctaggtcagtcccctcccctctcaactgggagcgtaagacttttcagaattgtaattttaatttaagcagcctgttacgttatgttcaggctgagagtttgttttgtaataatatcgatgcttccaaagtgtatggcaggtgctttggtagtatttcagttgataagtttgctgtaccccgaagtaggcaagttgatttacagcttggtaactctggatttctgcagactgctaattataagattgatacagctgccacttcgtgtcagctgcattacaccttgcctaagaataatgtcaccataaacaaccataacccctcgtcttggaataggaggtatggctttaatgatgctggcgtctttggcaaaaaccaacatgacgttgtttacgctcagcaatgttttactgtaagatctagttattgcccgtgtgctcaaccggacatagttagcccttgcactactcagactaagcctaagtctgcttttgttaatgtgggtgaccattgtgaaggcttaggtgttttagaagataattgtggcaatgctgatccacataagggttgtatctgtgccaacaattcatttattggatggtcacatgatacctgccttgttaatgatcgctgccaaatttttgctaatatattgttaaatggcattaatagtggtaccacatgttccacagatttgcagttgcctaatactgaagtggttactggcatttgtgtcaaatatgacctctacggtattactggacaaggtgtttttaaagaggttaaggctgactattataatagctggcaaacccttctgtatgatgttaatggtaatttgaatggttttcgtgatcttaccactaacaagacttatacgataaggagctgttatagtggccgtgtttctgctgcatttcataaagatgcacccgaaccggctctgctctatcgtaatataaattgtagctatgtttttagcaataatatttcccgtgaggagaacccacttaattactttgatagttatttgggttgtgttgttaatgctgataaccgcacggatgaggcgcttcctaattgtgatctccgtatgggtgctggcttatgcgttgattattcaaaatcacgcagggctcaccgatcagtttctactggctatcggttaactacatttgagccatacactccgatgttagttaatgatagtgtccaatccgttgatggattatatgagatgcaaataccaaccaattttactattgggcaccatgaggagttcattcaaactagatctccaaaggtgactatagattgtgctgcatttgtctgtggtgataacactgcatgcaggcagcagttggttgagtatggctctttctgtgttaatgttaatgccattcttaatgaggttaataacctcttggataatatgcaactacaagttgctagtgcattaatgcagggtgttactataagctcgagactgccagacggcatctcaggccctatagatgacattaattttagtcctctacttggatgcataggttcaacatgtgctgaagacggcaatggacctagtgcaatccgagggcgttctgctatagaggatttgttatttgacaaggtcaaattatctgatgttggctttgtcgaggcttataataattgcaccggtggtcaagaagttcgtgacctcctttgtgtacaatcttttaacggcatcaaagtattacctcctgtgttgtcagagagtcagatctctggctacacaaccggtgctactgcggcagctatgttcccaccgtggtcagcagctgccggtgtgccatttagtttaagtgttcaatatagaattaatggtttaggtgtcactatgaatgtgcttagtgagaaccaaaagatgattgctagtgcttttaacaatgcgctgggtgctatccaggatgggtttgatgcaaccaattctgctttaggtaagatccagtccgttgttaatgcaaatgctgaagcactcaataacttactaaatcaactttctaacaggtttggtgctattagtgcttctttacaagaaattctaactcggcttgaggctgtagaagcaaaagcccagatagatcgtcttattaatggcaggttaactgcacttaatgcgtatatatccaagcaacttagtgatagtacgcttattaaagttagtgctgctcaggccatagaaaaggtcaatgagtgcgttaagagccaaaccacgcgtattaatttctgtggcaatggtaatcatatattatctcttgtccagaatgcgccttatggcttatattttatacacttcagctatgtgccaatatcctttacaaccgcaaatgtgagtcctggactttgcatttctggtgatagaggattagcacctaaagctggatattttgttcaagatgatggagaatggaagttcacaggcagttcatattactaccctgaacccattacagataaaaacagtgtcattatgagtagttgcgcagtaaactacacaaaggcacctgaagttttcttgaacacttcaatacctaatccacccgactttaaggaggagttagataaatggtttaagaatcagacgtctattgcgcctgatttatctctcgatttcgagaagttaaatgttactttgctggacctgacgtatgagatgaacaggattcaggatgcaattaagaagttaaatgagagctacatcaacctcaaggaagttggcacatatgaaatgtatgtgaaatggccttggtatgtttggttgctaattggattagctggtgtagctgtttgtgtgttgttattctttatatgttgctgcacaggttgtggctcatgttgttttaagaagtgtggaaattgttgtgatgagtatggaggacaccaggacagtattgtgatacataatatttcctctcatgaggattgactatcacagcccctgcaggaaagacagaaaatctaaacaatttatagcattctcattgctacctggccccgtaagaggcagtcatagctatggccgtgttggtcctaaggctacattggctgctgtctttattggtccatttattgtagcatgtatgctaggcattggcctagtttatttattgcaattgcaagttcaaatttttcatgttaaggataccatacgtgtgactggcaagccagccactgtgtcttatactacaagtacaccagtaacaccgagcgcgacgacgctcgatggtactacgtatactttaattagacccactagctcttatacaagagtttatcttggtactccaagaggttttgattatagtacatttgggcctaagaccctagattatgttactaatctaaacctcatcttaattctggtcgtccatatacttttaaggcattgtccaggcatatgagaccaacagccacatggatttggcatgtgagtgatgcatggttacgccgcacgcgggactttggtgtcattcgcctagaagatttttgttttcaatttaattatagccaaccccgagttggttattgtagagttcctttaaaggcttggtgtagcaaccagggtaaatttgcagcgcagtttaccctaaaaagttgcgaaaaaccaggtcacgaaaaatttattactagcttcacggcctacggcagaactgtccaacaggccgttagcaagttagtagaagaagctgttgattttattctttttagggccacgcagctcgaaagaaatgtttaatttattccttacagacacagtatggtatgtggggcagattatttttatattcgcagtgtgtttgatggtcaccataattgtggttgccttccttgcgtctatcaaactttgtattcaactttgcggtttatgtaatactttggtgctgtccccttctatttatttgtatgataggagtaagcagctttataagtattataatgaagaaatgagactgcccctattagaggtggatgatatctaatccaaacattatgagtagtactactcaggccccagagcccgtctatcaatggacggccgacgaggcagttcaattccttaaggaatggaacttctcgttgggcattatactactctttattactatcatactacagttcggttacacgagccgtagcatgtttatttatgttgtgaaaatgataatcttgtggttaatgtggccactgactattgttttgtgtattttcaattgcgtgtatgcgctaaataatgtgtatcttggattttctatagtgtttactatagtgtccattgtaatctggattatgtattttgttaatagcataaggttgtttatcaggactggtagctggtggagcttcaaccccgaaacaaacaaccttatgtgtatagatatgaaaggtaccgtgtatgttagacccattattgaggattaccatacactaacagccactattattcgtggccacctctacatgcaaggtgttaagctaggcaccggtttctctttgtctgacttgcccgcttatgttacagttgctaaggtgtcacacctttgcacttataagcgcgcattcttagacaaggtagacggtgttagcggttttgctgtttatgtgaagtccaaggtcggaaattaccgactgccctcaaacaaaccgagtggcgcggacaccgcattgttgagaacctaatctaaactttaaggatgtcttttgttcctgggcaagaaaatgccggtggcagaagctcctctgtaaaccgcgctggtaatggaatcctcaagaagaccacttgggctgaccaaaccgagcgtggaccaaataatcaaaatagaggcagaaggaatcagccaaagcagactgcaactactcaacccaactccgggagtgtggttccccattactcctggttttctggcattacccagttccaaaagggaaaggagtttcagtttgcagaaggacaaggagtgcctattgccaatggaatccccgcttcagagcaaaagggatattggtatagacacaaccgccgttcttttaaaacacctgatgggcagcagaagcaattactgcccagatggtatttttactatcttggcacagggccccatgctggagccagttatggagacagcattgaaggtgtcttctgggttgcaaacagccaagcggacaccaatacccgctctgatattgtcgaaagggacccaagcagtcatgaggctattcctactaggtttgcgcccggcacggtattgcctcagggcttttatgttgaaggctctggaaggtctgcacctgctagccgatctggttcgcggtcacaatcccgtgggccaaataatcgcgctagaagcagttccaaccagcgccagcctgcctctactgtaaaacctgatatggccgaagaaattgctgctcttgttttggctaagctcggtaaagatgccggccagcccaagcaagtaacgaagcaaagtgccaaagaagtcaggcagaaaattttaaacaagcctcgccaaaagaggactccaaacaagcagtgcccagtgcagcagtgttttggaaagagaggccccaatcagaattttggaggctctgaaatgttaaaacttggaactagtgatccacagttccccattcttgcagagttggctccaacagttggtgccttcttctttggatctaaattagaattggtcaaaaagaattctggtggtgctgatgaacccaccaaagatgtgtatgagctgcaatattcaggtgcagttagatttgatagtactctacctggttttgagactatcatgaaagtgttgaatgagaatttgaatgcctaccagaaggatggtggtgcagatgtggtgagcccaaagccccaaagaaaagggcgtagacaggctcaggaaaagaaagatgaagtagataatgtaagcgttgcaaagcccaaaagctctgtgcagcgaaatgtaagtagagaattaaccccagaggatagaagtctgttggctcagatccttgatgatggcgtagtgccagatgggttagaagatgactctaatgtgtaaagagaatgaatcctatgtcggcgctcggtggtaacccctcgcgagaaagtcgggataggacactctctatcagaatggatgtcttgctgtcataacagatagagaaggttgtggcagaccctgtatcaattagttgaaagagattgcaaaatagagaatgtgtgagagaagttagcaaggtcctacgtctaaccataagaacggcgataggcgccccctgggaagagctcacatcagggtactattcctgcaatgccctagtaaatgaatgaagttgatcatggccaattggaagaatcacaaaaaaaaaaaaaaaaaaaaaaaaaaaaaaa